SEQUENCE LISTING (12 sequences)

SEQ ID NO: 1 - protein, 120 aa, Artificial Sequence
  Synthetic polypeptide
QVQLVQSGAEVKKPGASVKVSCKVSGYTLTELSIHWVRQAPGKGLEWMGGFDPEENEIVYAQRFQGRVTMTEDTSTDTAYMELSSLRSEDTAVYYCAIVGSFSPLTLGLWGQGTMVTVSS

SEQ ID NO: 2 - protein, 111 aa, Artificial Sequence
  Synthetic polypeptide
QSVLTQPPSVSGAPGQRVTISCTGSGSNIGAPYDVSWYQQLPGTAPKLLIYHNNKRPSGVPDRFSGSKSGTSASLAITGLQAEDEADYYCATVEAGLSGSVFGGGTKLTVL

SEQ ID NO: 3 - protein, 5 aa, Artificial Sequence
  Synthetic polypeptide
ELSIH

SEQ ID NO: 4 - protein, 17 aa, Artificial Sequence
  Synthetic polypeptide
GFDPEENEIVYAQRFQG

SEQ ID NO: 5 - protein, 11 aa, Artificial Sequence
  Synthetic polypeptide
VGSFSPLTLGL

SEQ ID NO: 6 - protein, 14 aa, Artificial Sequence
  Synthetic polypeptide
TGSGSNIGAPYDVS

SEQ ID NO: 7 - protein, 7 aa, Artificial Sequence
  Synthetic polypeptide
HNNKRPS

SEQ ID NO: 8 - protein, 11 aa, Artificial Sequence
  Synthetic polypeptide
ATVEAGLSGSV

SEQ ID NO: 9 - DNA, 5430 bp, Homo sapiens
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagagagtggaggatgctttttatacattggtgagagagatccgacaatacagattgaaaaaaatcagcaaagaagaaaagactcctggctgtgtgaaaattaaaaaatgcattataatgtaatctgggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 10 - DNA, 5306 bp, Homo sapiens
ctaggcggcggccgcggcggcggaggcagcagcggcggcggcagtggcggcggcgaaggtggcggcggctcggccagtactcccggcccccgccatttcggactgggagcgagcgcggcgcaggcactgaaggcggcggcggggccagaggctcagcggctcccaggtgcgggagagaggcctgctgaaaatgactgaatataaacttgtggtagttggagctggtggcgtaggcaagagtgccttgacgatacagctaattcagaatcattttgtggacgaatatgatccaacaatagaggattcctacaggaagcaagtagtaattgatggagaaacctgtctcttggatattctcgacacagcaggtcaagaggagtacagtgcaatgagggaccagtacatgaggactggggagggctttctttgtgtatttgccataaataatactaaatcatttgaagatattcaccattatagagaacaaattaaaagagttaaggactctgaagatgtacctatggtcctagtaggaaataaatgtgatttgccttctagaacagtagacacaaaacaggctcaggacttagcaagaagttatggaattccttttattgaaacatcagcaaagacaagacagggtgttgatgatgccttctatacattagttcgagaaattcgaaaacataaagaaaagatgagcaaagatggtaaaaagaagaaaaagaagtcaaagacaaagtgtgtaattatgtaaatacaatttgtacttttttcttaaggcatactagtacaagtggtaatttttgtacattacactaaattattagcatttgttttagcattacctaatttttttcctgctccatgcagactgttagcttttaccttaaatgcttattttaaaatgacagtggaagtttttttttcctctaagtgccagtattcccagagttttggtttttgaactagcaatgcctgtgaaaaagaaactgaatacctaagatttctgtcttggggcttttggtgcatgcagttgattacttcttatttttcttaccaattgtgaatgttggtgtgaaacaaattaatgaagcttttgaatcatccctattctgtgttttatctagtcacataaatggattaattactaatttcagttgagaccttctaattggtttttactgaaacattgagggaacacaaatttatgggcttcctgatgatgattcttctaggcatcatgtcctatagtttgtcatccctgatgaatgtaaagttacactgttcacaaaggttttgtctcctttccactgctattagtcatggtcactctccccaaaatattatattttttctataaaaagaaaaaaatggaaaaaaattacaaggcaatggaaactattataaggccatttccttttcacattagataaattactataaagactcctaatagcttttcctgttaaggcagacccagtatgaaatggggattattatagcaaccattttggggctatatttacatgctactaaatttttataataattgaaaagattttaacaagtataaaaaattctcataggaattaaatgtagtctccctgtgtcagactgctctttcatagtataactttaaatcttttcttcaacttgagtctttgaagatagttttaattctgcttgtgacattaaaagattatttgggccagttatagcttattaggtgttgaagagaccaaggttgcaaggccaggccctgtgtgaacctttgagctttcatagagagtttcacagcatggactgtgtccccacggtcatccagtgttgtcatgcattggttagtcaaaatggggagggactagggcagtttggatagctcaacaagatacaatctcactctgtggtggtcctgctgacaaatcaagagcattgcttttgtttcttaagaaaacaaactcttttttaaaaattacttttaaatattaactcaaaagttgagattttggggtggtggtgtgccaagacattaattttttttttaaacaatgaagtgaaaaagttttacaatctctaggtttggctagttctcttaacactggttaaattaacattgcataaacacttttcaagtctgatccatatttaataatgctttaaaataaaaataaaaacaatccttttgataaatttaaaatgttacttattttaaaataaatgaagtgagatggcatggtgaggtgaaagtatcactggactaggaagaaggtgacttaggttctagataggtgtcttttaggactctgattttgaggacatcacttactatccatttcttcatgttaaaagaagtcatctcaaactcttagttttttttttttacaactatgtaatttatattccatttacataaggatacacttatttgtcaagctcagcacaatctgtaaatttttaacctatgttacaccatcttcagtgccagtcttgggcaaaattgtgcaagaggtgaagtttatatttgaatatccattctcgttttaggactcttcttccatattagtgtcatcttgcctccctaccttccacatgccccatgacttgatgcagttttaatacttgtaattcccctaaccataagatttactgctgctgtggatatctccatgaagttttcccactgagtcacatcagaaatgccctacatcttatttcctcagggctcaagagaatctgacagataccataaagggatttgacctaatcactaattttcaggtggtggctgatgctttgaacatctctttgctgcccaatccattagcgacagtaggatttttcaaacctggtatgaatagacagaaccctatccagtggaaggagaatttaataaagatagtgctgaaagaattccttaggtaatctataactaggactactcctggtaacagtaatacattccattgttttagtaaccagaaatcttcatgcaatgaaaaatactttaattcatgaagcttactttttttttttggtgtcagagtctcgctcttgtcacccaggctggaatgcagtggcgccatctcagctcactgcaacctccatctcccaggttcaagcgattctcgtgcctcggcctcctgagtagctgggattacaggcgtgtgccactacactcaactaatttttgtatttttaggagagacggggtttcaccctgttggccaggctggtctcgaactcctgacctcaagtgattcacccaccttggcctcataaacctgttttgcagaactcatttattcagcaaatatttattgagtgcctaccagatgccagtcaccacacaaggcactgggtatatggtatccccaaacaagagacataatcccggtccttaggtagtgctagtgtggtctgtaatatcttactaaggcctttggtatacgacccagagataacacgatgcgtattttagttttgcaaagaaggggtttggtctctgtgccagctctataattgttttgctacgattccactgaaactcttcgatcaagctactttatgtaaatcacttcattgttttaaaggaataaacttgattatattgtttttttatttggcataactgtgattcttttaggacaattactgtacacattaaggtgtatgtcagatattcatattgacccaaatgtgtaatattccagttttctctgcataagtaattaaaatatacttaaaaattaatagttttatctgggtacaaataaacaggtgcctgaactagttcacagacaaggaaacttctatgtaaaaatcactatgatttctgaattgctatgtgaaactacagatctttggaacactgtttaggtagggtgttaagacttacacagtacctcgtttctacacagagaaagaaatggccatacttcaggaactgcagtgcttatgaggggatatttaggcctcttgaatttttgatgtagatgggcatttttttaaggtagtggttaattacctttatgtgaactttgaatggtttaacaaaagatttgtttttgtagagattttaaagggggagaattctagaaataaatgttacctaattattacagccttaaagacaaaaatccttgttgaagtttttttaaaaaaagctaaattacatagacttaggcattaacatgtttgtggaagaatatagcagacgtatattgtatcatttgagtgaatgttcccaagtaggcattctaggctctatttaactgagtcacactgcataggaatttagaacctaacttttataggttatcaaaactgttgtcaccattgcacaattttgtcctaatatatacatagaaactttgtggggcatgttaagttacagtttgcacaagttcatctcatttgtattccattgattttttttttcttctaaacattttttcttcaaacagtatataactttttttaggggatttttttttagacagcaaaaactatctgaagatttccatttgtcaaaaagtaatgatttcttgataattgtgtagtaatgttttttagaacccagcagttaccttaaagctgaatttatatttagtaacttctgtgttaatactggatagcatgaattctgcattgagaaactgaatagctgtcataaaatgaaactttctttctaaagaaagatactcacatgagttcttgaagaatagtcataactagattaagatctgtgttttagtttaatagtttgaagtgcctgtttgggataatgataggtaatttagatgaatttaggggaaaaaaaagttatctgcagatatgttgagggcccatctctccccccacacccccacagagctaactgggttacagtgttttatccgaaagtttccaattccactgtcttgtgttttcatgttgaaaatacttttgcatttttcctttgagtgccaatttcttactagtactatttcttaatgtaacatgtttacctggaatgtattttaactatttttgtatagtgtaaactgaaacatgcacattttgtacattgtgctttcttttgtgggacatatgcagtgtgatccagttgttttccatcatttggttgcgctgacctaggaatgttggtcatatcaaacattaaaaatgaccactcttttaattgaaattaacttttaaatgtttataggagtatgtgctgtgaagtgatctaaaatttgtaatatttttgtcatgaactgtactactcctaattattgtaatgtaataaaaatagttacagtgac

SEQ ID NO: 11 - protein, 189 aa, Homo sapiens [sequence omitted]

SEQ ID NO: 12 - protein, 188 aa, Homo sapiens [sequence omitted]

(19) United States
(12) Patent Application Publication
(10) Pub. No.: US 2022/0184179 A1
(43) Pub. Date: Jun. 16, 2022

(54) TREATMENT OF CANCERS WITH GM-CSF ANTAGONISTS

(71) Applicant: Kiniksa Pharmaceuticals, Ltd., Hamilton (BM)

(72) Inventors: Joe Pirrello, Lexington, MA (US); Eben Tessari, Lexington, MA (US); Luis Carvajal, Lexington, MA (US); Annalisa D'andrea, Lexington, MA (US)

(21) Appl. No.: 17/511,183

(22) Filed: Oct. 26, 2021

Related U.S. Application Data
(60) Provisional application No. 63/242,849, filed on Sep. 10, 2021.
(60) Provisional application No. 63/120,319, filed on Dec. 2, 2020.
(60) Provisional application No. 63/105,773, filed on Oct. 26, 2020.

(51) Int. Cl. A61K 38/17; C07K 16/28; A61K 45/06; A61P 35/00

(52) U.S. Cl. CPC A61K 38/1793; C07K 16/2866; C07K 2317/76; A61P 35/00; A61K 45/06

(57) ABSTRACT

The present invention provides, among other things, a method of treating cancer comprising administering a GM-CSF antagonist to the patient in need of treatment, wherein the administration of the GM-CSF antagonist improves, stabilizes or reduces one or more symptoms of the cancer in the patient. The present invention also provides, among other things, a method of inhibiting progression of cancer in a patient suffering from cancer with one or more mutations in KRAS, NRAS and/or JAK2 comprising administering a GM-CSF antagonist to the patient.

### CROSS-REFERENCE TO RELATED APPLICATION

This application claims priority to U.S. provisional application No. 63/105,773, filed on Oct. 26, 2020, U.S. provisional application No. 63/120,319 filed on Dec. 2, 2020, and U.S. provisional application No. 63/242,849 filed on Sep. 10, 2021, the contents of each of which is hereby incorporated by reference in its entirety.

### SEQUENCE LISTING

The present specification makes reference to a Sequence Listing (submitted electronically as a .txt file named “KPL-035BWO_ST25.txt” on Oct. 26, 2021). The .txt file was generated on Oct. 26, 2021 and is 21 KB in size. The entire contents of the Sequence Listing are herein incorporated by reference.

### BACKGROUND

Tumor cells express unique antigens that are potentially recognized by the host T cell repertoire and serve as potent targets for tumor immunotherapy. However, tumor cells evade host immunity and express inhibitory cytokines that suppress native antigen presenting effector cell populations. One element in this immunosuppressive milieu is the increased presence of regulatory T cells that are found in the tumor bed, draining lymph nodes, and in the circulation of patients with malignancy. One area of further investigation is the development of therapeutics to reverse tumor-associated energy and to stimulate effector cells to recognize and eliminate malignant cells.

### SUMMARY OF THE INVENTION

The present invention provides, among other things, an improved method for treating cancer using a GM-CSF antagonist. NRAS, KRAS, JAK2 and PTPN11 genes, among other genes, play a significant role in signaling pathways and mutations in these genes have been associated with various cancers. For example, KRAS is one of the most challenging targets in cancer. One KRAS (Kirsten rat sarcoma viral oncogene homolog) mutation is present in up to 25% of all human tumors, and this is one of the most frequently activated oncogenes. Despite its discovery more than 60 years, researchers still struggle to inhibit its mutated form. The present invention provides, among other things, an improved method for treating cancer with one or more mutations in KRAS, NRAS, JAK2 and/or PTPN11 using a GM-CSF antagonist. The present invention is based, in part, on a surprising discovery that GM-CSF induces expression of PD-L1 on MDSCs that have immunosuppressive activity and that this expression can be suppressed by antagonizing GM-CSF. The present invention also provides methods for treating cancer using a GM-CSF antagonist in combination with other cancer therapies as further described herein. The present invention further provides methods for treating chronic myelomonocytic leukemia (CMML) and juvenile myelomonocytic leukemia (JMML) using a GM-CSF antagonists.

In some aspects, the present invention provides a method of treating cancer with a KRAS mutation comprising administering a GM-CSF antagonist to the patient in need of treatment, wherein the administration of the GM-CSF antagonist results in inhibition of an immunosuppressive activity of myeloid-derived suppressor cells (MDSCs).

In some aspects, the present invention provides a method of treating cancer comprising administering a GM-CSF antagonist to the patient in need of treatment, wherein the administration of the GM-CSF antagonist results in inhibition of an immunosuppressive activity of myeloid-derived suppressor cells (MDSCs).

In some aspects, the present invention provides a method of inhibiting immunosuppressive activity of myeloid-derived suppressor cells (MDSCs) in a patient suffering from cancer with one or more mutations in KRAS, NRAS, JAK2, and/or PTPN11 comprising administering a GM-CSF antagonist to the patient.

In some aspects, the present invention provides a method of inhibiting immunosuppressive activity of myeloid-derived suppressor cells (MDSCs) in a patient suffering from cancer comprising administering a GM-CSF antagonist to the patient.

In some aspects, the present invention provides a method of enhancing immune response for cancer treatment comprising administering a GM-CSF antagonist to a patient receiving a cancer treatment, wherein the patient is suffering from cancer with one or more mutations in KRAS, NRAS, JAK2, and/or PTPN11, wherein the immune response is increased as compared to a control.

In some aspects, the present invention provides a method of enhancing immune response for cancer treatment comprising administering a GM-CSF antagonist to a patient receiving a cancer treatment, wherein the immune response is increased as compared to a control.

In some embodiments, the cancer with one or more mutations in KRAS, NRAS, JAK2, and/or PTPN11 is chronic myelomonocytic leukemia (CMML).

In some embodiments, the cancer with one or more mutations in KRAS, NRAS, JAK2, and/or PTPN11 is juvenile myelomonocytic leukemia (JMML).

In some embodiments, the immune response is a percentage of T cell proliferation. In some embodiments, T cells are CD8 positive (CD8+). In some embodiments, T cells are CD4 positive (CD4+). In some embodiments, T cells are double-positive for CD8 and CD4 (CD8+/CD4+).

In some embodiments, the control is indicative of the immune response level in the patient prior to the administration of GM-CSF antagonist. In some embodiments, the control is a reference immune response level in a control patient receiving the cancer treatment without GM-CSF antagonist or a reference immune response level based on historical data.

In some embodiments, the cancer therapy is an immunotherapy.

In some embodiments, the administering the GM-CSF antagonist increases the efficacy of the immunotherapy.

In one aspect, the present invention provides, among other things, a method of suppressing PD-L1 in a patient suffering from cancer with one or more mutations in KRAS, NRAS, JAK2, and/or PTPN11, comprising administering a GM-CSF antagonist to a patient in need of treatment as compared to a control.

In one aspect, the present invention provides, among other things, a method of suppressing PD-L1 in a cancer patient comprising administering a GM-CSF antagonist to a patient in need of treatment as compared to a control.

In some embodiments, the administering the GM-CSF antagonist decreases a level of PD-L1 in the patient.

In some embodiments, the control is indicative of the PD-L1 level in the patient prior to the administration of GM-CSF antagonist.

In some embodiments, the control is a reference PD-L1 level in a control patient receiving the cancer treatment without GM-CSF antagonist or a reference PD-L1 level based on historical data.

In some embodiments, the level of PD-L1 in the patient is decreased by at least 10% as compared to the control. In some embodiments, the level of PD-L1 in the patient is decreased by at least 15% as compared to the control. In some embodiments, the level of PD-L1 in the patient is decreased by at least 20% as compared to the control. In some embodiments, the level of PD-L1 in the patient is decreased by at least 30% as compared to the control. In some embodiments, the level of PD-L1 in the patient is decreased by at least 40% as compared to the control. In some embodiments, the level of PD-L1 in the patient is decreased by at least 45% as compared to the control. In some embodiments, the level of PD-L1 in the patient is decreased by at least 50% as compared to the control. In some embodiments, the level of PD-L1 in the patient is decreased by at least 60% as compared to the control. In some embodiments, the level of PD-L1 in the patient is decreased by at least 70% as compared to the control. In some embodiments, the level of PD-L1 in the patient is decreased by at least 75% as compared to the control. In some embodiments, the level of PD-L1 in the patient is decreased by at least 80% as compared to the control. In some embodiments, the level of PD-L1 in the patient is decreased by at least 85% as compared to the control. In some embodiments, the level of PD-L1 in the patient is decreased by at least 90% as compared to the control.

In some embodiments, the PD-L1 is expressed on MDSCs. In some embodiments, the PD-L1 is expressed on circulating MDSCs. In some embodiments, the PD-L1 is expressed on plasma-derived MDSCs. In some embodiments, the PD-L1 is expressed on tumor cells. In some embodiments, PD-L1 is expressed on tumor-infiltrating immune cells.

In some embodiments, the patient has circulating myeloid derived suppressor cells (MDSCs).

In some embodiments, the patient suffers from a cancer with a low level of infiltrating T cells.

In some embodiments, the patient suffers from an immune checkpoint inhibitor (ICI) refractory cancer.

In some embodiments, the patient suffers from a late stage or metastatic cancer.

In some embodiments, the patient suffers from a cancer selected from non-small cell lung cancer (NSCLC), colorectal cancer (CRC), pancreatic cancer, colon adenocarcinoma, rectal adenocarcinoma, endometrial carcinoma, adenocarcinoma, appendix Adenocarcinoma, acute myeloid leukemia, breast cancer, or ovarian cancer.

In some embodiments, the patient suffers from a cancer selected from non-small cell lung cancer (NSCLC), colorectal cancer (CRC), or pancreatic cancer.

In some embodiments, the patient suffers from a cancer selected from breast cancer, colorectal cancer (CRC), prostate cancer, melanoma, bladder carcinoma, pancreatic cancer, pancreatic ductal adenocarcinoma, hepatocellular carcinoma, gastric cancer, non-small cell lung cancer (NSCLC), small cell lung cancer (SCLC), head and neck squamous cell carcinoma, non-Hodgkin lymphoma, cervical cancer, gastrointestinal cancer, urogenital cancer, brain cancer, mesothelioma, renal cell cancer, gynecological cancer, ovarian cancer, endometrial cancer, lung cancer, gastrointestinal cancer, pancreatic cancer, oesophageal cancers, hepatocellular cancer, cholangiocellular cancer, brain cancers, mesothelioma, malignant melanoma, Merkel Cell Carcinoma, multiple myeloma, acute myelogenous leukemia, chronic myelogenous leukemia, myelodysplastic syndrome or acute lymphoblastic leukemia.

In some embodiments, the patient suffers from a cancer selected from Stage IV breast cancer, Stage IV colorectal cancer (CRC), prostate cancer, or melanoma.

The method of any one of the preceding claims, wherein the method further comprises administering at least one other cancer therapy to the patient.

In some embodiments, the at least one other cancer therapy is chemotherapy, MDSC-targeted therapy, immunotherapy, radiation therapy and combinations thereof.

In some embodiments, the GM-CSF antagonist and the other cancer therapy are administered concurrently.

In some embodiments, the GM-CSF antagonist and the other cancer therapy are administered sequentially.

In some embodiments, the patient has received a treatment with the other cancer therapy prior to the administration of the GM-CSF antagonist.

In some embodiments, the patient has received a treatment with the GM-CSF antagonist prior to the administration of the other cancer therapy.

In some embodiments, the other cancer therapy is an ICI.

In some embodiments, the ICI antagonizes the activity of PD-1, CTLA-4, B7, BTLA, HVEM, TIM-3, GAL-9, LAG3, VISTA, KIR, 2B4, CD160, CGEN-15049, CHK1, CHK2, or A2aR.

In some embodiments, the ICI is selected from an anti-PD-1 antibody (optionally pembrolizumab, nivolumab, cemiplimab), an anti-PD-L1 antibody (optionally atezolizumab, avelumab, durvalumab), an anti-CTLA-4 antibody (optionally ipillimumab), an anti-PD-L2 antibody, an anti-B7-H3 antibody, an anti-B7-H4 antibody, an anti-BTLA antibody, an anti-HVEM antibody, an anti-TIM-3 antibody, an anti-GAL-9 antibody, an anti-LAG3 antibody, an anti-VISTA antibody, an anti-MR antibody, an anti-2B4 antibody, an anti-CD160 antibody, an anti-CGEN-15049 antibody, an anti-CHK1 antibody, an anti-CHK2 antibody, an anti-A2aR antibody, an anti-B-7 antibody, and combinations thereof.

In some embodiments, the ICI is an anti-PD-L1 antibody. In some embodiments, the ICI is an anti-PD-L1 antibody.

In some embodiments, the method further comprises administering a chemotherapy agent to the patient.

In some embodiments, the MDSC-targeted therapy is selected from an anti-CFS-1R antibody, an anti-IL-6 antibody, all-trans retinoic acid, axitinib, entinostat, gemcitabine, or phenformin, and combinations thereof.

In some embodiments, the immunotherapy is selected from a monoclonal antibody, cytokine, cancer vaccine, T-cell engaging therapies, and combinations thereof.

In some embodiments, the monoclonal antibody is selected from an anti-CD3 antibody, an anti-CD52 antibody, an anti-PD1 antibody, an anti-PD-Ll antibody, an anti-CTLA4 antibody, an anti-CD20 antibody, an anti-BCMA antibody, bi-specific antibodies, or bispecific T-cell engager (BiTE) antibodies, and combinations thereof.

In some embodiments, the cytokines are selected from IFNa, IFNp, IFNy, IFN, IL-2, IL-7, IL-15, IL-21, IL-11, IL-12, IL-18, hGM-CSF, TNFa, or any combination thereof.

In some embodiments, the GM-CSF antagonist is an anti-GM-CSF antibody or a fragment thereof.

In some embodiments, the GM-CSF antagonist is a soluble GM-CSF receptor.

In some embodiments, the GM-CSF antagonist is an anti-GM-CSF receptor antibody or a fragment thereof.

In some embodiments, the anti-GM-CSF receptor antibody or a fragment thereof is an anti-GM-CSFRα antibody or a fragment thereof.

In some embodiments, the anti-GM-CSFRα antibody or a fragment thereof is a monoclonal antibody specific for human GM-CSFRα.

In some embodiments, the anti-GM-CSFRα antibody is human or humanized IgG4 antibody.

In some embodiments, the anti-GM-CSFRα antibody is mavrilimumab.

In some embodiments, the anti-GM-CSFRα antibody a fragment thereof comprises a light chain complementary-determining region 1 (LCDR1) defined by SEQ ID NO: 6, a light chain complementary-determining region 2 (LCDR2) defined by SEQ ID NO: 7, and a light chain complementary-determining region 3 (LCDR3) defined by SEQ ID NO: 8; and a heavy chain complementary-determining region 1 (HCDR1) defined by SEQ ID NO: 3, a heavy chain complementary-determining region 2 (HCDR2) defined by SEQ ID NO: 4, and a heavy chain complementary-determining region 3 (HCDR3) defined by SEQ ID NO: 5.

In some embodiments, the administration of the GM-CSF antagonist and/or the ICI results in reduced level of MDSCs in the patient as compared to a control.

In some embodiments, the administration of the GM-CSF antagonist and/or the ICI results in reduced level of MDSC-mediated immunosuppressive activity in the patent as compared to a control.

In some embodiments, the administration of the GM-CSF antagonist and/or the ICI results in reduced percentage of Lin-CD14+HLA-DR-M-MDSCs in the peripheral blood of the patient as compared to a control.

In some embodiments, the administration of the GM-CSF antagonist and/or the ICI results in increased percentage of mature MDSC cells in the patient as compared to a control.

In some embodiments, the administration of the GM-CSF antagonist and/or the ICI results in a reduced level of Treg cells, macrophages, and/or neutrophils as compared to a control.

In some embodiments, the administration of the GM-CSF antagonist and/or the ICI results in a decreased level of an inhibitory cytokine.

In some embodiments, the inhibitory cytokine is selected from IL-10 and TGFβ.

In some embodiments, the administration of the GM-CSF antagonist and/or the ICI results in a decreased level of an immune suppressive factor.

In some embodiments, the immune suppressive factor is selected from arginase 1, inducible nitric oxide synthase (iNOS), peroxynitrite, nitric oxide, reactive oxygen species, tumor associated macrophages, and combinations thereof.

In some embodiments, the administration of the GM-CSF antagonist and/or the ICI results in an increased level of CD4+ T effector cells as compared to a control.

In some embodiments, the control is a pre-treatment level or percentage in the patient, or a reference level or percentage based on historical data.

In some aspects, the present invention provides a pharmaceutical composition for treating cancer with one or more mutations in KRAS, NRAS, JAK2, and/or PTPN11 comprising a GM-CSF antagonist and an ICI.

In some aspects, the present invention provides a pharmaceutical composition for treating cancer comprising a GM-CSF antagonist and an ICI.

In some embodiments, the GM-CSF antagonist is an anti-GM-CSF antibody or a fragment thereof.

In some embodiments, the GM-CSF antagonist is a soluble GM-CSF receptor.

In some embodiments, the GM-CSF antagonist is an anti-GM-CSF receptor antibody or a fragment thereof.

In some embodiments, the anti-GM-CSF receptor antibody or a fragment thereof is an anti-GM-CSFRα antibody or a fragment thereof.

In some embodiments, the anti-GM-CSFRα antibody or a fragment thereof is a monoclonal antibody specific for human GM-CSFRα.

In some embodiments, the anti-GM-CSFRα antibody is human or humanized IgG4 antibody.

In some embodiments, the anti-GM-CSFRα antibody is mavrilimumab.

In some embodiments, the anti-GM-CSFRα antibody a fragment thereof comprises a light chain complementary-determining region 1 (LCDR1) defined by SEQ ID NO: 6, a light chain complementary-determining region 2 (LCDR2) defined by SEQ ID NO: 7, and a light chain complementary-determining region 3 (LCDR3) defined by SEQ ID NO: 8; and a heavy chain complementary-determining region 1 (HCDR1) defined by SEQ ID NO: 3, a heavy chain complementary-determining region 2 (HCDR2) defined by SEQ ID NO: 4, and a heavy chain complementary-determining region 3 (HCDR3) defined by SEQ ID NO: 5.

In some embodiments, the ICI antagonizes the activity of PD-1, CTLA-4, B7, BTLA, HVEM, TIM-3, GAL-9, LAG3, VISTA, KIR, 2B4, CD160, CGEN-15049, CHK1, CHK2, A2aR, and combinations thereof.

In some embodiments, the ICI is selected from an anti-PD-1 antibody (optionally pembrolizumab, nivolumab, cemiplimab), an anti-PD-L1 antibody (optionally atezolizumab, avelumab, durvalumab), an anti-CTLA-4 antibody (optionally ipillimumab), an anti-PD-L2 antibody, an anti-B7-H3 antibody, an anti-B7-H4 antibody, an anti-BTLA antibody, an anti-HVEM antibody, an anti-TIM-3 antibody, an anti-GAL-9 antibody, an anti-LAG3 antibody, an anti-VISTA antibody, an anti-MR antibody, an anti-2B4 antibody, an anti-CD160 antibody, an anti-CGEN-15049 antibody, an anti-CHK1 antibody, an anti-CHK2 antibody, an anti-A2aR antibody, an anti-B-7 antibody, and combinations thereof.

In some aspects, the present invention provides a kit for treating cancer with a KRAS mutation comprising a pharmaceutical composition comprising a GM-CSF antagonist and pharmaceutical composition comprising at least one other cancer therapy selected from a chemotherapy, MDSC-targeted therapy, immunotherapy, radiation therapy and combinations thereof.

In some aspects, the present invention provides a kit for treating cancer comprising a pharmaceutical composition comprising a GM-CSF antagonist and pharmaceutical composition comprising at least one other cancer therapy selected from a chemotherapy, MDSC-targeted therapy, immunotherapy, radiation therapy and combinations thereof.

In some embodiments, the immunotherapy is an ICI selected from an anti-PD-1 antibody (optionally pembrolizumab, nivolumab, cemiplimab), an anti-PD-L1 antibody (optionally atezolizumab, avelumab, durvalumab), an anti-CTLA-4 antibody (optionally ipillimumab), an anti-PD-L2 antibody, an anti-B7-H3 antibody, an anti-B7-H4 antibody, an anti-BTLA antibody, an anti-HVEM antibody, an anti-TIM-3 antibody, an anti-GAL-9 antibody, an anti-LAG3 antibody, an anti-VISTA antibody, an anti-MR antibody, an anti-2B4 antibody, an anti-CD160 antibody, an anti-CGEN-15049 antibody, an anti-CHK1 antibody, an anti-CHK2 antibody, an anti-A2aR antibody, an anti-B-7 antibody, and combinations thereof.

In some aspects, the present invention provides, among other things, a method of treating cancer with increased activation of KRAS signaling pathway as compared to a normal, comprising administering a GM-CSF antagonist to the patient in need of treatment, wherein the administration of the GM-CSF antagonist results in inhibition of an immunosuppressive activity of myeloid-derived suppressor cells (MDSCs).

In some aspects, the present invention provides a method of inhibiting immunosuppressive activity of myeloid-derived suppressor cells (MDSCs) in a patient suffering from cancer with increased activation of KRAS signaling pathway as compared to normal, comprising administering a GM-CSF antagonist to the patient.

In some aspects, the present invention provides a method of enhancing immune response for cancer treatment comprising administering a GM-CSF antagonist to a patient receiving a cancer treatment, wherein the patient is suffering from cancer with increased activation of KRAS signaling pathway as compared to normal, wherein the immune response is increased as compared to a control.

In one aspect, the present invention provides, among other things, a method of suppressing PD-L1 in a patient suffering from cancer with increased activation of KRAS signaling pathway as compared to normal, comprising administering a GM-CSF antagonist to a patient in need of treatment as compared to a control.

In some aspects, the present invention provides a pharmaceutical composition for treating cancer with increased activation of KRAS signaling pathway as compared to normal, comprising a GM-CSF antagonist and an ICI.

In some aspects, the present invention provides a kit for treating cancer with increased activation of KRAS signaling pathway as compared to normal, comprising a pharmaceutical composition comprising a GM-CSF antagonist and pharmaceutical composition comprising at least one other cancer therapy selected from a chemotherapy, MDSC-targeted therapy, immunotherapy, radiation therapy and combinations thereof.

In some embodiments, a normal level of KRAS signaling pathway activation is the level of KRAS signaling pathway activation in a healthy, non-cancerous patient, or a tissue sample obtained from the healthy, non-cancerous patient. In some embodiments, a normal level of KRAS signaling pathway activation is the level of KRAS signaling pathway activation in a non-KRAS mutant cancer patient, or a tissue sample obtained from the non-KRAS mutant cancer patient. In some embodiments, a normal level of KRAS signaling pathway activation is the level of KRAS signaling pathway activation in the non-cancerous tissue of a KRAS mutant cancer patient. In some embodiments, a normal level of KRAS signaling pathway activation is the level of KRAS signaling pathway activation in a non-cancerous cells.

In some embodiments, one or more mutations that result in increased activation of KRAS signaling pathway are in a gene encoding a protein involved in KRAS signaling pathway. In some embodiments, one or more mutations that result in increased activation of KRAS signaling pathway are in a KRAS gene. In some embodiments, one or more mutations that result in increased activation of KRAS signaling pathway are in a gene encoding a protein involved in signaling pathway upstream of KRAS signaling pathway. In some embodiments, one or more mutations that result in increased activation of KRAS signaling pathway are in a gene encoding a protein involved in signaling pathway downstream of KRAS signaling pathway. In some embodiments, one or more mutations that result in increased activation of KRAS signaling pathway are oncogenic mutations.

In some aspects, the present invention provides a method of enhancing immune response for cancer treatment comprising administering a GM-CSF antagonist to a patient receiving a cancer treatment, wherein the patient is suffering from cancer with increased activation of NRAS signaling pathway as compared to normal, wherein the immune response is increased as compared to a control.

In some aspects, the present invention provides a method of enhancing immune response for cancer treatment comprising administering a GM-CSF antagonist to a patient receiving a cancer treatment, wherein the patient is suffering from cancer with perturbed JAK2 signaling pathway as compared to normal, wherein the immune response is increased as compared to a control.

In some aspects, the present invention provides a method of enhancing immune response for cancer treatment comprising administering a GM-CSF antagonist to a patient receiving a cancer treatment, wherein the patient is suffering from cancer with perturbed PTPN11 signaling pathway as compared to normal, wherein the immune response is increased as compared to a control.

In some aspects, the present invention provides a method of treating cancer in a patient comprising administering to the patient in need of treatment a therapeutically effective dose of a GM-CSF antagonist, wherein the administration improves, stabilizes reduces one or more symptoms of the cancer as compared to a control, wherein the cancer is chronic myelomonocytic leukemia (CMML) or juvenile myelomonocytic leukemia (JMML).

In some embodiments, the cancer has one or more mutations in KRAS, NRAS, PTPN11 or JAK2.

In some embodiments, the CMML is categorized as MPN-CMML. In some embodiments, the CMML is categorized as MDS-CMML.

In some embodiments, the CMML is classified as CMML-0. In some embodiments, the CMML is classified as CMML-1. In some embodiments, the CMML is classified as CMML-2.

In some embodiments, the administration of a GM-CSF antagonist to a patient with CMML reduces the time to transformation to AML. In some embodiments, the administration of a GM-CSF antagonist to a patient with CMML prevents the transformation to AML.

In some embodiments, the administration of a GM-CSF antagonist to a patient with JMML reduces the time to transformation to AML. In some embodiments, the administration of a GM-CSF antagonist to a patient with JMML prevents the transformation to AML

In some embodiments, the GM-CSF antagonist is selected from the group consisting of mavrilimumab, namilumab, otilimab, lenzilumab, gimsilumab and TJM2. In some embodiments, the GM-CSF antagonist is mavrilimumab. In some embodiments, the GM-CSF antagonist is namilumab. In some embodiments, the GM-CSF antagonist is otilimab. In some embodiments, the GM-CSF antagonist is lenzilumab. In some embodiments, the GM-CSF antagonist is gimsilumab. In some embodiments, the GM-CSF antagonist is TJM2.

### BRIEF DESCRIPTION OF THE DRAWING

The drawings are for illustration purposes only, not for limitation.

FIG. 1 is an exemplary bar graph illustrating T cell proliferation assay with CD14+ cells (MDSCs) from blood of patient with pancreatic cancer. T cell proliferation is suppressed following co-culture of healthy donor allogeneic T cells and CD14+ cells sorted from blood of pancreatic cancer patients, compared with those T cells cultured in complete media alone. T cell proliferation is rescued following culture with an anti-GM-CSFRα antibody and CD14+ cells sorted from blood of pancreatic cancer patients.

FIG. 2 is an exemplary graph illustrating GM-CSF expression levels in different cancer cell lines, as measured by amount of human GM-CSF secreted into cancer cell supernatants (n=2). Plotted are the means of two independent ELISA readings performed in duplicate, error bars show the standard deviation (+/−).

FIG. 3 is a series of exemplary bar graphs illustrating that cancer cell-conditioned medium can polarize monocytes to phenotypic MDSCs (CD14+ cells). To generate tumor-conditioned media (CM), four different cell lines were plated and cultured according to methods known in the art. CD14+ monocytes cells were then cultured in the presence of CM for 6 days and analyzed for gene and protein expression. Low levels of HLA-DR biomarker is indicative of MDSC phenotype. An increase in phenotypic MDSCs was observed when CD14+ monocytes were incubated with conditioned medium from GM-CSF-expressing cancer cells, as compared to CD+14 cells that were grown in normal culture medium (control).

FIG. 4 is a series of exemplary bar graphs illustrating the PD-L1 expression on MDSCs cultured with various media. Data show that cancer cell-conditioned medium (CM) and CM supplemented with recombinant GM-CSF can induce expression of PD-L1 on MDSCs. Additionally, anti-GM-CSFRα antibody (Ab) can reduce PD-L1 expression levels on MDSCs.

FIG. 5A and FIG. 5B are s a series of exemplary bar graphs illustrating the PD-L1 expression on MDSCs cultured with various media. Data show that cancer cell-conditioned medium (CM) and CM supplemented at Day 1 with recombinant GM-CSF can induce expression of PD-L1 on MDSCs compared to MDSCs that were grown in normal culture medium (Medium). Additionally, anti-GM-CSFRα antibody (Ab) can reduce PD-L1 level on MDSCs grown in CM. Data in FIG. 5A show PD-L1 expression when CM and an anti-GM-CSFRα antibody (Ab) are added concurrently. PD-L1 expression was measured after 3 days of treatment. Data in FIG. 5B show PD-L1 expression when an anti-GM-CSFRα antibody is added 72 hours after culturing with CM. PD-L1 expression was measured after 24 hours of treatment with the anti-GM-CSFRα antibody.

FIG. 6A is a series of exemplary bar graphs illustrating proliferation of CD8+ T-cells cultured with CD14+ monocytes from healthy controls (n=3) with or without cancer cell conditioned medium for 3 days (+/−100 μg/mL anti-GM-CSFRα antibody). On day 3, CD3+ T cells were labelled with CFSE, stimulated with CD3/CD28/IL-2 and co-cultured with monocytes for 5 days. Proliferation was monitored by measuring CFSE dilution and normalized to proliferating T-cells co-cultured with monocytes in corresponding cell culture medium. FIG. 6B is a series of exemplary bar graphs illustrating T cell proliferation with monocytes treated with conditioned medium from GM-CSF expressing cancer cell lines with and without supplemental human recombinant GM-CSF and/or an anti-GM-CSFRα antibody (Ab). Monocytes were cultured in conditioned medium from GM-CSF expressing cancer cell lines (CM) for three days. T cells (1+10⁵ cells) were prepared by labeling with 0.1 μM CFSE and stimulation with 10 ng/mL of IL-2 and 10 μL of soluble CD3/CD28 T cell activator (ImmunoCult) in IMDM cell culture medium. Then, the stimulated T cells were co-cultured with CM-treated monocytes (at a ratio of 2:1 monocyte: T cell) with or without recombinant GM-CSF (10 ng/mL) and/or anti-GM-CSFRα antibody (100 μg/mL) in a mix lymphocyte reaction (MLR). Stimulated T-cells in IMDM culture medium together with healthy monocytes were used as a control. T-cells were expanded for 5 days, collected and stained for CD4 and CD8, which are markers for helper T and cytotoxic T cells. Cell proliferation was measured by flow cytometry and evaluated by CFSE dilution. Left panel shows the results of the T-cell proliferation assay in terms of % of cells proliferating and right panel shows the results in terms of % of max (MFI) (signal detection of CFSE dilution in CD4+ or CD8+ cells) by flow cytometry. FIG. 6C is an exemplary graph of proliferation of CD8+ T-cells cultured with CD14+ monocytes from healthy controls (n=3) with or without colorectal cancer cell conditioned medium for 3 days. Anti-GM-CSFRα antibody was added to the monocyte culture on day 0, while anti-PD-L1 antibody was added to MLR on day 3. CD3+ T cells were labelled with CFSE, stimulated with CD3/CD28/IL-2 and co-cultured with monocytes for 5 days. Proliferation was monitored by measuring CFSE dilution and normalized to proliferating T-cells co-cultured with monocytes in corresponding cell culture medium.

FIG. 7A is an exemplary workflow of the study to examine the polarization of CD14+ monocytes to phenotypic MDSCs with KRAS-mutant cancer cell conditioned medium. CD14=monocytes were incubated with either base medium or conditioned medium from cancer cells for 3 days, followed by phenotypic analysis by flow cytometry. FIG. 7B is a series of representative histogram overlays showing degreased expression of HLA=DR and increased expression of CD11b and PD-L1. FIG. 7C is a series of exemplary bar graphs illustrating relative expression, plotted as the geometric mean fluorescence intensity (gMFI), of HLA-DR, CD11b, and PD-L1 in healthy donor monocytes cultured with cancer cell conditioned medium. P-values were generated using a two-tailed, paired comparison student's test.

FIG. 8A and FIG. 8B are s a series of exemplary graphs illustrating the PD-L1 expression on MDSCs cultured with various media and anti-GM-CSFRα antibody. FIG. 8A is representative graphs showing relative expression of PD-L1 plotted as the geometric mean fluorescence intensity (gMFI) in healthy donor monocytes cultured with conditioned medium from colorectal and pancreatic cancer cells with or without anti-GM-CSFRα antibody at various concentrations. FIG. 8B is a representative graph showing relative expression of PD-L1 plotted as the geometric mean fluorescence intensity (gMFI) in healthy donor monocytes cultured with conditioned medium from cervical cancer cells. P-values were generated using ordinary one-way ANOVA with Dunnett's multiple comparisons of the means. (CM=conditioned medium; n.s=not significant)

FIG. 9A and FIG. 9B are a series of exemplary graphs illustrating mean tumor volume in mice with pancreatic carcinoma. FIG. 9A is representative graphs showing mean tumor volume in mice for each treatment group 3 days after the end of the treatment. FIG. 9B is representative graphs showing mean tumor volume in mice for each treatment group 12 days after the end of the treatment.

FIG. 10 is an exemplary Kaplan-Meier plot for the time to tumor volume of 2000 mm³ compared with a log-rank test. The time to tumor volume of 2000 mm³ was longer in mice treated with GM-CSF antagonist alone or in combination with anti-PD-1 antibody compared to a control group.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

Antibody: As used herein, the term “antibody” refers to immunoglobulin molecules and immunologically active portions of immunoglobulin (Ig) molecules, i.e., molecules that contain an antigen binding site that binds (immunoreacts with) an antigen. By “binds” or “immunoreacts with” is meant that the antibody reacts with one or more antigenic determinants of the desired. Antibodies include, antibody fragments. Antibodies also include, but are not limited to, polyclonal, monoclonal, chimeric dAb (domain antibody), single chain, Fab, Fab′, F(ab′)2 fragments, scFvs, and Fab expression libraries. An antibody may be a whole antibody, or immunoglobulin, or an antibody fragment.

Amino acid: As used herein, term “amino acid,” in its broadest sense, refers to any compound and/or substance that can be incorporated into a polypeptide chain. In some embodiments, an amino acid has the general structure H₂N—C(H)(R)—COOH. In some embodiments, an amino acid is a naturally occurring amino acid. In some embodiments, an amino acid is a synthetic amino acid; in some embodiments, an amino acid is a d-amino acid; in some embodiments, an amino acid is an 1-amino acid. “Standard amino acid” refers to any of the twenty standard 1-amino acids commonly found in naturally occurring peptides. “Nonstandard amino acid” refers to any amino acid, other than the standard amino acids, regardless of whether it is prepared synthetically or obtained from a natural source. As used herein, “synthetic amino acid” encompasses chemically modified amino acids, including but not limited to salts, amino acid derivatives (such as amides), and/or substitutions. Amino acids, including carboxyl- and/or amino-terminal amino acids in peptides, can be modified by methylation, amidation, acetylation, protecting groups, and/or substitution with other chemical groups that can change the peptide's circulating half-life without adversely affecting their activity. Amino acids may participate in a disulfide bond. Amino acids may comprise one or posttranslational modifications, such as association with one or more chemical entities (e.g., methyl groups, acetate groups, acetyl groups, phosphate groups, formyl moieties, isoprenoid groups, sulfate groups, polyethylene glycol moieties, lipid moieties, carbohydrate moieties, biotin moieties, etc.). The term “amino acid” is used interchangeably with “amino acid residue,” and may refer to a free amino acid and/or to an amino acid residue of a peptide. It will be apparent from the context in which the term is used whether it refers to a free amino acid or a residue of a peptide.

Amelioration: As used herein, the term “amelioration” is meant the prevention, reduction or palliation of a state, or improvement of the state of a subject. Amelioration includes, but does not require complete recovery or complete prevention of a disease condition. In some embodiments, amelioration includes increasing levels of relevant protein or its activity that is deficient in relevant disease tissues.

Approximately or about: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Delivery: As used herein, the term “delivery” encompasses both local and systemic delivery.

Improve, increase, inhibit or reduce: As used herein, the terms “improve,” “increase” “inhibit” or “reduce,” or grammatical equivalents, indicate values that are relative to a baseline measurement, such as a measurement in the same individual prior to initiation of the treatment described herein, or a measurement in a control or control subject (or multiple control subject) in the absence of the treatment described herein, e.g., a subject who is administered a placebo. A “control” or “control subject” is a subject afflicted with the same form of disease as the subject being treated, who is about the same age as the subject being treated.

“Inhibition” or “inhibiting”: As used herein “inhibition” or “inhibiting,” or grammatical equivalents, means reduction, decrease or inhibition of biological activity. Neutralization: As used herein, neutralization means reduction or inhibition of biological activity of the protein to which the neutralizing antibody binds, in this case GM-CSFRα, e.g. reduction or inhibition of GM-CSF binding to GM-CSFRα, or of signaling by GM-CSFRα e.g. as measured by GM-CSFRα-mediated responses. The reduction or inhibition in biological activity may be partial or total. The degree to which an antibody neutralizes GM-CSFRα is referred to as its neutralizing potency.

“KRAS mutation”: As used herein, “KRAS mutation”, “KRAS mutant”, “K-Ras mutation”, “K-Ras mutant”, or grammatical equivalents means one or more mutation in a KRAS gene or protein as compared to the wild-type sequence. The wild-type or normal KRAS gene suppresses tumor growth, but the mutant KRAS gene promotes cell proliferation, and causes tumor generation. “Cancer with a KRAS mutation” or “KRAS mutant cancer” is (a) a cancer cell or tumor cell containing a somatic KRAS mutation, or (b) a cancer cell or tumor cell with an abnormal expression level of KRAS including, but not limited to, amplification of the KRAS encoding DNA, or over-expression of the KRAS gene, when compared to level found in normal, non-cancer cells. As used herein, “cancer with increased activation of KRAS signaling pathway” is a cancer or tumor cell with one or more mutations in a gene that result in aberrant activation of the KRAS signaling pathway. In some embodiments, one or more mutations that result in increased activation of KRAS signaling pathway are in a gene encoding a protein involved in KRAS signaling pathway. In some embodiments, one or more mutations that result in increased activation of KRAS signaling pathway are in a KRAS gene. In some embodiments, one or more mutations that result in increased activation of KRAS signaling pathway are in a gene encoding a protein involved in signaling pathway upstream of KRAS signaling pathway. In some embodiments, one or more mutations that result in increased activation of KRAS signaling pathway are in a gene encoding a protein involved in signaling pathway downstream of KRAS signaling pathway. In some embodiments, one or more mutations that result in increased activation of KRAS signaling pathway are oncogenic mutations.

Patient: As used herein, the term “patient” refers to any organism to which a provided composition may be administered, e.g., for experimental, diagnostic, prophylactic, cosmetic, and/or therapeutic purposes. Typical patients include animals (e.g., mammals such as mice, rats, rabbits, non-human primates, and/or humans). In some embodiments, a patient is a human. A human includes pre- and post-natal forms.

Pharmaceutically acceptable: The term “pharmaceutically acceptable” as used herein, refers to substances that, within the scope of sound medical judgment, are suitable for use in contact with the tissues of human beings and animals without excessive toxicity, irritation, allergic response, or other problem or complication, commensurate with a reasonable benefit/risk ratio.

Substantial identity: The phrase “substantial identity” is used herein to refer to a comparison between amino acid or nucleic acid sequences. As will be appreciated by those of ordinary skill in the art, two sequences are generally considered to be “substantially identical” if they contain identical residues in corresponding positions. As is well known in this art, amino acid or nucleic acid sequences may be compared using any of a variety of algorithms, including those available in commercial computer programs such as BLAS TN for nucleotide sequences and BLASTP, gapped BLAST, and PSI-BLAST for amino acid sequences. Exemplary such programs are described in Altschul, et al., Basic local alignment search tool, J Mal. Biol., 215 (3): 403-410, 1990; Altschul, et al., Methods in Enzymology; Altschul et al., Nucleic Acids Res. 25:3389-3402, 1997; Baxevanis et al., Bioinformatics: A Practical Guide to the Analysis of Genes and Proteins, Wiley, 1998; and Misener, et al., (eds.), Bioinformatics Methods and Protocols (Methods in Molecular Biology, Vol. 132), Humana Press, 1999. In addition to identifying identical sequences, the programs mentioned above typically provide an indication of the degree of identity. In some embodiments, two sequences are considered to be substantially identical if at least 50%, 55%, 60%, 65%, 70%, 75%, 80%, 85%, 90%, 91%, 92%, 93%, 94%, 95%, 96%, 97%, 98%, 99% or more of their corresponding residues are identical over a relevant stretch of residues. In some embodiments, the relevant stretch is a complete sequence. In some embodiments, the relevant stretch is at least 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 425, 450, 475, 500 or more residues.

Subject: As used herein, the term “subject” refers to a human or any non-human animal (e.g., mouse, rat, rabbit, dog, cat, cattle, swine, sheep, horse or primate). A human includes pre- and post-natal forms. In many embodiments, a subject is a human being. A subject can be a patient, which refers to a human presenting to a medical provider for diagnosis or treatment of a disease. The term “subject” is used herein interchangeably with “individual” or “patient.” A subject can be afflicted with or is susceptible to a disease or disorder but may or may not display symptoms of the disease or disorder.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Systemic distribution or delivery: As used herein, the terms “systemic distribution,” “systemic delivery,” or grammatical equivalent, refer to a delivery or distribution mechanism or approach that affect the entire body or an entire organism. Typically, systemic distribution or delivery is accomplished via body's circulation system, e.g., blood stream. Compared to the definition of “local distribution or delivery.”

Therapeutically effective amount: As used herein, the term “therapeutically effective amount” of a therapeutic agent means an amount that is sufficient, when administered to a subject suffering from or susceptible to a disease, disorder, and/or condition, to treat, diagnose, prevent, and/or delay the onset of the symptom(s) of the disease, disorder, and/or condition. It will be appreciated by those of ordinary skill in the art that a therapeutically effective amount is typically administered via a dosing regimen comprising at least one unit dose.

Treating: As used herein, the term “treat,” “treatment,” or “treating” refers to any method used to partially or completely alleviate, ameliorate, relieve, inhibit, prevent, delay onset of, reduce severity of and/or reduce incidence of one or more symptoms or features of a particular disease, disorder, and/or condition. Treatment may be administered to a subject who does not exhibit signs of a disease and/or exhibits only early signs of the disease for the purpose of decreasing the risk of developing pathology associated with the disease.

### DETAILED DESCRIPTION

The present invention provides, among other things, method of treating cancer in a patient in need of treatment using a GM-CSF antagonist. In certain embodiments, the present invention provides methods of treating cancer by inhibiting immunosuppressive activity of myeloid-derived suppressor cells (MDSCs) in a patient in need of treatment using a GM-CSF antagonist. In some embodiments, a GM-CSF antagonist is used in combination with an immune checkpoint inhibitor. It is contemplated that the present invention is particularly effective in treating immune checkpoint inhibitory (ICI) refractory or resistant cancers, or late stage or metastatic cancers.

Various aspects of the invention are described in detail in the following sections. The use of sections is not meant to limit the invention. Each section can apply to any aspect of the invention. In this application, the use of “or” means “and/or” unless stated otherwise.

### KRAS (Kirsten Rat Sarcoma Viral Oncogene Homolog) Mutation

The KRAS (Kirsten rat sarcoma viral oncogene homolog) gene provides instructions for making a protein called K-Ras that is part of a signaling pathway known as the RAS/MAPK pathway. The protein relays signals from outside the cell to the cell's nucleus. These signals instruct the cell to grow and divide (proliferate) or to mature and take on specialized functions (differentiate). The K-Ras protein is a GTPase, which means it converts a molecule called GTP into another molecule called GDP. In this way the K-Ras protein acts like a switch that is turned on and off by the GTP and GDP molecules. To transmit signals, it must be turned on by attaching (binding) to a molecule of GTP. The K-Ras protein is turned off (inactivated) when it converts the GTP to GDP. When the protein is bound to GDP, it does not relay signals to the cell's nucleus.

The KRAS gene belongs to a class of genes known as oncogenes. Oncogenes are normal genes with an important role in the process of stimulation of controlled cellular proliferation. Mutations in these genes result in uncontrolled proliferation and development of cancer. RAS genes are expressed in normal cells, and are involved in controlled cell growth. Three distinct mutations of RAS have been identified: H-ras, N-ras, and K-ras. In general, colon, pancreas and lung carcinomas have mutations of KRAS, bladder tumors have HRAS mutations, and hematopoietic neoplasms are associated with NRAS mutations.

Mutation in KRAS can be related to malignant tumors, such as lung adenocarcinoma, mucinous adenoma, ductal carcinoma of the pancreas, and colorectal carcinoma. In human colorectal cancer, KRAS mutation appears to induce overexpression of GST-π via activation of AP-1. See, e.g., Miyanishi et al., Gastroenterology, 2001; 121 (4):865-74.

Mutant KRAS is found in colon cancer (Burmer G C, Loeb L A, 1989, Proc. Natl. Acad. Sci. U.S.A., 86 (7):2403-2407), pancreatic cancer (Almoguera C, et al., 1988, Cell, 53 (4):549-554) and lung cancer (Tam I Y, et al., 2006, Clin. Cancer Res., 12 (5):1647-1653). KRAS accounts for 90% of RAS mutations in lung adenocarcinomas (Forbes S, et al. Cosmic 2005. Br J Cancer, 2006; 94:318-322).

KRAS gene may also be amplified in colorectal cancer. KRAS amplification can be mutually exclusive with KRAS mutations. See, e.g., Valtorta E, et al., 2013, Int. J. Cancer, 133 (5):1259-65. Amplification of wild-type KRAS also has been observed in ovarian, gastric, uterine, and lung cancers. See, e.g., Chen Y, et al., 2014, PLoS ONE, 9 (5):e98293.

The human KRAS gene sequence has two preferred transcript variants, having the nucleic acid sequences given in GenBank Accession Nos. NM_033360 (transcript variant a) (SEQ ID NO: 9) and NM_004985.5 (transcript variant b) (SEQ ID NO: 10). The human KRAS protein sequence has two preferred variants, having the amino acid sequence given in GenBank Accession Nos. NP_203524 (isoform a) (SEQ ID NO: 11) and NP_004976.2 (isoform b) (SEQ ID NO: 12).

In some embodiments, cancer with a KRAS mutation is a cancer cell or tumor cell containing an oncogenic KRAS mutation. Oncogenic KRAS mutations associated with cancer include, without limitation, G12A, G12S, G12D, G12V, G13D, G12C, Q61R, Q61L, Q61K, G12R, and G12C. In some embodiments, KRAS mutations include oncogenic substitution of G12 and/or G13 that leads to constitutive activation of KRAS. The skilled artisan will understand that a KRAS gene comprising a KRAS mutation other than those identified above, and/or combinations of the KRAS mutations above, and/or other KRAS mutations that preferably lead to constitutive activation of KRAS, are also an oncogenic KRAS mutation encompassed by the present invention. In some embodiments, cancer with a KRAS mutation is a cancer cell or tumor cell containing a somatic KRAS mutation. In some embodiments, cancer with a KRAS mutation is a cancer cell or tumor cell with a constitutive activation of KRAS signaling pathway including, but not limited to, amplification of the KRAS encoding DNA, or over-expression of the KRAS gene when compared to level found in normal, non-cancer cells.

In one aspect, the present invention provides, among other things, a method of treating cancer with increased activation of KRAS signaling pathway with GM-CSF antagonist. As used herein, “cancer with increased activation of KRAS signaling pathway” is a cancer or tumor cell with one or more mutations in a gene that result in aberrant activation of the KRAS signaling pathway. In some embodiments, one or more mutations that result in increased activation of KRAS signaling pathway are in a gene encoding a protein involved in KRAS signaling pathway. In some embodiments, one or more mutations that result in increased activation of KRAS signaling pathway are in a KRAS gene. In some embodiments, one or more mutations that result in increased activation of KRAS signaling pathway are in a gene encoding a protein involved in signaling pathway upstream of KRAS signaling pathway. In some embodiments, one or more mutations that result in increased activation of KRAS signaling pathway are in a gene encoding a protein involved in signaling pathway downstream of KRAS signaling pathway. In some embodiments, one or more mutations that result in increased activation of KRAS signaling pathway as compared to normal are oncogenic mutations.

In some embodiments, one or mutations that result in increased activation of KRAS signaling pathway as compared to normal are in gene encoding a protein involved in RAF signaling pathway. In some embodiments, one or mutations that result in increased activation of KRAS signaling pathway as compared to normal are in gene encoding a protein involved in MAPK signaling pathway. In some embodiments, one or mutations that result in increased activation of KRAS signaling pathway as compared to normal are in gene encoding a protein involved in PI3K signaling pathway. In some embodiments, one or mutations that result in increased activation of KRAS signaling pathway as compared to normal are in gene encoding a protein involved in AKT signaling pathway. In some embodiments, one or mutations that result in increased activation of KRAS signaling pathway as compared to normal are in gene encoding a protein involved in RAL signaling pathway. In some embodiments, one or mutations that result in increased activation of KRAS signaling pathway as compared to normal are in gene encoding a protein involved in Wnt signaling pathway. In some embodiments, one or mutations that result in increased activation of KRAS signaling pathway as compared to normal are in gene encoding a protein involved in receptor tyrosine kinase (RTK) signaling pathway. In some embodiments, one or mutations that result in increased activation of KRAS signaling pathway as compared to normal are in gene encoding a protein involved in mTOR signaling pathway. In some embodiments, one or mutations that result in increased activation of KRAS signaling pathway as compared to normal are in gene encoding a protein involved in MEK signaling pathway. In some embodiments, one or mutations that result in increased activation of KRAS signaling pathway as compared to normal are in gene encoding a protein involved in ERK signaling pathway. In some embodiments, one or mutations that result in increased activation of KRAS signaling pathway as compared to normal are in gene encoding a protein involved in NF-kB signaling pathway.

In some embodiments, a KRAS mutation is a one or more mutation in the KRAS gene or KRAS protein that promotes cell proliferation, causes tumor generation, or leads to amplification of the KRAS encoding DNA, or over-expression of the KRAS gene, deficiency of KRAS protein, aberrant overexpression, or activation of KRAS protein when compared to level found in normal, non-cancer cells. In some embodiments, one or more mutation includes deletion, insertion, or substitution of one or more nucleotides or amino acids in a gene or protein compared to the wild-type gene or protein. In some embodiments, a KRAS mutation is an amino acid substitution at residue G12 in KRAS protein. In some embodiments, a KRAS mutation is an amino acid substitution at residue G13 in KRAS protein. In some embodiments, KRAS mutations include oncogenic substitution of G12 and/or G13 that lead to constitutive activation of KRAS. In some embodiments, a KRAS mutation is an amino acid substitution at residue Q61 in KRAS protein. In some embodiments, a KRAS mutation is a G12D mutation in the KRAS protein. In some embodiments, a KRAS mutation is a G12V mutation in the KRAS protein. In some embodiments, a KRAS mutation is a G13D mutation in the KRAS protein. In some embodiments, a KRAS mutation is a G12C mutation in the KRAS protein. In some embodiments, a KRAS mutation is a Q61R mutation in the KRAS protein. In some embodiments, a KRAS mutation is a Q61L mutation in the KRAS protein. In some embodiments, a KRAS mutation is a Q61K mutation in the KRAS protein. In some embodiments, a KRAS mutation is a G12R mutation in the KRAS protein. In some embodiments, a KRAS mutation is a G12C mutation in the KRAS protein. In some embodiments, a KRAS mutation is a G12A mutation in the KRAS protein. In some embodiments, a KRAS mutation is a G12S mutation in the KRAS protein.

In some embodiments, cancer with a KRAS mutation has elevated levels of GM-CSF as compared to a control. In some embodiments, elevated levels of GM-CSF is detected in a patient sample. In some embodiments, a patient sample is a serum sample. In some embodiments, a patient sample is a tissue sample. In some embodiments, a patient sample is from tumor biopsy. In some embodiments, a control is a level of GM-CSF in a healthy subject without cancer. In some embodiments, a control is a level of GM-CSF in a healthy tissue sample from the patient. In some embodiments, a patient is selected based on the level of GM-CSF at baseline. In some embodiments, a patient has an elevated level of GM-CSF as compared to a control.

### NRAS (Neuroblastoma RAS Viral Oncogene Homolog) Mutation

The NRAS protein activates the Ras, Raf, MAP protein kinase/extracellular signal-regulated kinase (MEK), extracellular signal-regulated kinase (ERK) pathway (Ras/Raf/MEK/ERK pathway) in cells. This pathway is downstream of the EGFR receptor, and plays a central role in the regulation of a variety of cellular functions dependent upon cellular context, including cellular proliferation, differentiation, survival, immortalization, invasion and angiogenesis (reviewed in Peyssonnaux and Eychene, Biology of the Cell, 2001, 93:3-62). Indeed, the Ras-dependent Raf-MEK-MAPK cascade is one of the key signaling pathways responsible for conveying both mitogenic and invasive signals from the cell surface to the nucleus resulting in changes in gene expression and cell fate.

In some embodiments, cancer with a NRAS mutation is a cancer cell or tumor cell containing an oncogenic NRAS mutation. Oncogenic NRAS mutations associated with cancer include, without limitation, G12A, G12S, G12D, G12V, G13D, G12C, E63R, E63L, E63K, G12R, and G12C. In some embodiments, NRAS mutations include oncogenic substitution of G12 and/or E63 that leads to constitutive activation of NRAS. The skilled artisan will understand that a NRAS gene comprising a NRAS mutation other than those identified above, and/or combinations of the NRAS mutations above, and/or other NRAS mutations that preferably lead to constitutive activation of NRAS, are also an oncogenic NRAS mutation encompassed by the present invention. In some embodiments, cancer with a NRAS mutation is a cancer cell or tumor cell containing a somatic NRAS mutation. In some embodiments, cancer with a NRAS mutation is a cancer cell or tumor cell with a constitutive activation of NRAS signaling pathway including, but not limited to, amplification of the NRAS encoding DNA, or over-expression of the NRAS gene when compared to level found in normal, non-cancer cells.

In one aspect, the present invention provides, among other things, a method of treating cancer with increased activation of NRAS signaling pathway with GM-CSF antagonist. As used herein, “cancer with increased activation of NRAS signaling pathway” is a cancer or tumor cell with one or more mutations in a gene that result in aberrant activation of the NRAS signaling pathway. In some embodiments, one or more mutations that result in increased activation of NRAS signaling pathway are in a gene encoding a protein involved in NRAS signaling pathway. In some embodiments, one or more mutations that result in increased activation of NRAS signaling pathway are in a NRAS gene. In some embodiments, one or more mutations that result in increased activation of NRAS signaling pathway are in a gene encoding a protein involved in signaling pathway upstream of NRAS signaling pathway. In some embodiments, one or more mutations that result in increased activation of NRAS signaling pathway are in a gene encoding a protein involved in signaling pathway downstream of NRAS signaling pathway. In some embodiments, one or more mutations that result in increased activation of NRAS signaling pathway as compared to normal are oncogenic mutations.

In some embodiments, a NRAS mutation is a one or more mutation in the KRAS gene or NRAS protein that promotes cell proliferation, causes tumor generation, or leads to amplification of the NRAS encoding DNA, or over-expression of the NRAS gene, deficiency of NRAS protein, aberrant overexpression, or activation of NRAS protein when compared to level found in normal, non-cancer cells. In some embodiments, one or more mutation includes deletion, insertion, or substitution of one or more nucleotides or amino acids in a gene or protein compared to the wild-type gene or protein. In some embodiments, a NRAS mutation is an amino acid substitution at residue G12 in NRAS protein. In some embodiments, a KRAS mutation is an amino acid substitution at residue E63 in NRAS protein. In some embodiments, NRAS mutations include oncogenic substitution of G12 and/or E63 that lead to constitutive activation of NRAS. In some embodiments, a NRAS mutation is an amino acid substitution at residue G12 in NRAS protein. In some embodiments, a NRAS mutation is a G12D mutation in the NRAS protein. In some embodiments, a NRAS mutation is a G12V mutation in the NRAS protein. In some embodiments, a NRAS mutation is a G13D mutation in the KRAS protein. In some embodiments, a NRAS mutation is a G12C mutation in the NRAS protein. In some embodiments, a NRAS mutation is a E63R mutation in the NRAS protein. In some embodiments, a NRAS mutation is a E63L mutation in the NRAS protein. In some embodiments, a NRAS mutation is a E63K mutation in the NRAS protein. In some embodiments, a NRAS mutation is a G12R mutation in the NRAS protein. In some embodiments, a NRAS mutation is a G12C mutation in the NRAS protein. In some embodiments, a NRAS mutation is a G12A mutation in the NRAS protein. In some embodiments, a NRAS mutation is a G12S mutation in the NRAS protein.

In some embodiments, cancer with a NRAS mutation has elevated levels of GM-CSF as compared to a control. In some embodiments, elevated levels of GM-CSF is detected in a patient sample. In some embodiments, a patient sample is a serum sample. In some embodiments, a patient sample is a tissue sample. In some embodiments, a patient sample is from tumor biopsy. In some embodiments, a control is a level of GM-CSF in a healthy subject without cancer. In some embodiments, a control is a level of GM-CSF in a healthy tissue sample from the patient. In some embodiments, a patient is selected based on the level of GM-CSF at baseline. In some embodiments, a patient has an elevated level of GM-CSF as compared to a control.

### JAK2 (Janus Kinase 2) and PTPN11 Mutations

JAK2 gene codes for the non-receptor tyrosine kinase Janus kinase 2, which is a member of the Janus kinase family and has been implicated in signaling by members of the type II cytokine receptor family (e.g. interferon receptors), the GM-CSF receptor family (IL-3R, IL-5R and GM-CSF-R), the gp130 receptor family (e.g., IL-6R), and the single chain receptors (e.g. Epo-R, Tpo-R, GH-R, PRL-R). JAK2 signaling is activated downstream from the prolactin receptor. Other names for JAK2 are JTK10 and THCYT3. SHP2, the nonreceptor tyrosine phosphatase encoded by PTPN11, also participates in signaling events downstream of the receptors of growth factors, cytokines, hormones, antigens, and extracellular matrixes. JAK2 and SHP2 forms a complex signaling network in hematopoietic progenitor cells. Perturbed JAK2 and SHP2 signaling may induce hematopoietic malignancies.

Mutations in JAK2 have been identified in the majority of patients with Myeloproliferative neoplasms (MPNs), which are clonal disorders of hematopoietic stem cells with increased proliferation of myeloid cells and effective maturation, leading to peripheral blood leukocytosis and excess erythrocytes or platelets, underscoring the importance of constitutive activation of JAK2 signaling caused by mutations. More than 50% of patients with MPNs harbor the JAK2 V617F mutation. Additionally, mutations in PTPN11, the gene encoding the cytoplasmic protein with tyrosine phosphatase SHP-2, are a major molecular event in JMML. SHP-2 is a signal transducer that relays signals from activated growth factor and cytokine receptors to RAS and other intracellular signaling molecules and is required for sustained activation of the MAPK cascade. Accumulating evidence supports that PTPN11, NRAS, and KRAS2 mutations are largely mutually exclusive in JMML and other hematologic malignancies. On the other hand, PTPN11 defects were identified in only ˜1.3% of the CMML cases, indicated that PTPN11 lesions are rare genetic events in CMML. Exon 3, exon 8, and exon 13 in PTPN11 are known to be mutation hot spots, resulting in mutated SHP2 protein. The SHP2 mutations detected in JMML patients are G60R, D61Y, D61N, E69K, A72T, A72V, T73I, E76K, E76Q, E76G, E139D, and G506P.

In one aspect, the present invention provides, among other things, a method of treating cancer with increased activation or perturbation of JAK2 signaling pathway with GM-CSF antagonist. As used herein, “cancer with increased activation or perturbation of JAK2 signaling pathway” is a cancer or tumor cell with one or more mutations in a gene that result in aberrant activation of the JAK2 signaling pathway or in perturbation in downstream events (e.g., SHP2 signaling). In some embodiments, one or more mutations that result in increased activation of JAK2 signaling pathway are in a gene encoding a protein involved in JAK2 signaling pathway. In some embodiments, one or more mutations that result in perturbation of JAK2 signaling pathway are in a gene encoding a protein involved in JAK2 or SHP2 signaling pathway. In some embodiments, one or more mutations that result in increased activation of JAK2 signaling pathway are in a JAK2 gene. In some embodiments, one or more mutations that result perturbation of JAK2 signaling pathway are in a JAK2 gene. In some embodiments, one or more mutations that result perturbation of JAK2 signaling pathway are in a PTPN11 gene. In some embodiments, one or more mutations that result in increased activation of JAK2 signaling pathway are in a gene encoding a protein involved in signaling pathway upstream of JAK2 signaling pathway. In some embodiments, one or more mutations that result in increased activation of JAK2 signaling pathway are in a gene encoding a protein involved in signaling pathway downstream of JAK2 signaling pathway. In some embodiments, a gene encoding a protein involved in signaling pathway downstream of JAK2 signaling pathway is PTPN11. In some embodiments, one or more mutations that result in increased activation of JAK2 signaling pathway as compared to normal are oncogenic mutations.

In some embodiments, a JAK2 mutation is a one or more mutation in the JAK2 gene or JAK2 protein that disrupts JAK/STAT pathway. In some embodiments, one or more mutation includes deletion, insertion, or substitution of one or more nucleotides or amino acids in a gene or protein compared to the wild-type gene or protein. In some embodiments, a JAK2 mutation is an amino acid substitution at residue V617 in JAK2 protein. In some embodiments, a JAK2 mutation is a V617F mutation in the JAK2 protein.

In some embodiments, cancer with a JAK2 mutation has elevated levels of GM-CSF as compared to a control. In some embodiments, elevated levels of GM-CSF is detected in a patient sample. In some embodiments, a patient sample is a serum sample. In some embodiments, a patient sample is a tissue sample. In some embodiments, a patient sample is from tumor biopsy. In some embodiments, a control is a level of GM-CSF in a healthy subject without cancer. In some embodiments, a control is a level of GM-CSF in a healthy tissue sample from the patient. In some embodiments, a patient is selected based on the level of GM-CSF at baseline. In some embodiments, a patient has an elevated level of GM-CSF as compared to a control.

In some embodiments, SHP2 mutation is a one or more mutation in the PTPN11 gene or SHP2 protein that disrupts downstream events of JAK/STAT pathway. In some embodiments, one or more mutation includes deletion, insertion, or substitution of one or more nucleotides or amino acids in a gene or protein compared to the wild-type gene or protein. In some embodiments, a SHP2 mutation is an amino acid substitution at residue G60 in SHP2 protein. In some embodiments, a SHP2 mutation is an amino acid substitution at residue D61 in SHP2 protein. In some embodiments, a SHP2 mutation is an amino acid substitution at residue E69 in SHP2 protein. In some embodiments, a SHP2 mutation is an amino acid substitution at residue A72 in SHP2 protein. In some embodiments, a SHP2 mutation is an amino acid substitution at residue T73 in SHP2 protein. In some embodiments, a SHP2 mutation is an amino acid substitution at residue E76 in SHP2 protein. In some embodiments, a SHP2 mutation is an amino acid substitution at residue E139 in SHP2 protein. In some embodiments, a SHP2 mutation is an amino acid substitution at residue G506 in SHP2 protein. In some embodiments, a SHP mutation is a G60R mutation in the SHP2 protein. In some embodiments, a SHP mutation is a G60R mutation in the SHP2 protein. In some embodiments, a SHP2 mutation is a G60R mutation in the SHP2 protein. In some embodiments, a SHP2 mutation is a D61Y mutation in the SHP2 protein. In some embodiments, a SHP2 mutation is a D61N mutation in the SHP2 protein. In some embodiments, a SHP2 mutation is an E69K mutation in the SHP2 protein. In some embodiments, a SHP2 mutation is an A72T mutation in the SHP2 protein. In some embodiments, a SHP2 mutation is an A72V mutation in the SHP2 protein. In some embodiments, a SHP2 mutation is a T73I mutation in the SHP2 protein. In some embodiments, a SHP2 mutation is an E76K mutation in the SHP2 protein. In some embodiments, a SHP2 mutation is an E76Q mutation in the SHP2 protein. In some embodiments, a SHP2 mutation is an E76G mutation in the SHP2 protein. In some embodiments, a SHP2 mutation is an E139D mutation in the SHP2 protein. In some embodiments, a SHP2 mutation is a G506P mutation in the SHP2 protein.

### Myeloid-Derived Suppressor Cells (MDSCs)

MDSC are a heterogenous group of immune cells from the myeloid lineage. MDSCs strongly expand in pathological situations such as chronic infections and cancer and are distinguished from other myeloid cell types in which they possess strong immunosuppressive activities, rather than immunostimulatory properties. Monocytes that have diminished or no HLA-DR expression, called CD14³⁰ HLA-DR^{lo/neg} monocytes are grouped into MDSCs and can alter adaptive immunity and produce immunosuppression.

MDSCs accumulate in the peripheral blood, lymphoid organ, spleen, and tumor sites in cancer, infection, chronic inflammation, transplantation, and autoimmunity. The specific pathways by which tumors recruit, expand, and activate MDSCs remain unknown, but increasing evidence exists for the involvement of interleukin (IL-1β), IL-6, cyclooxyhenase 2 (COX2)-generated PGE₂, high concentrations of GM-CSF, M-CSF, vascular endothelial growth factor (VEGF), IL-10, transforming growth beta (TGFβ), indoleamine 2, 3-dioxyhenase (IDO), FLT3 ligand, and stem cell factor.

Co-culture of immune competent cells with tumor cell lines as been shown to induce tolerogenic DC or MDSC. Previous studies also suggest that tumor cells release GM-CSF, which induces granulocyte ROS production to inhibit T-cell function. Additionally, the expression of programmed death ligand 1 (PD-L1) is increased on the surface of MDSC in murine tumor models, though the role of this in MDSC-mediated suppression remains unclear.

### Programmed Death Ligand 1 (PD-L1)

Programmed death ligand 1 (PD-L1; also known as CD274) is an immune checkpoint protein that binds to its receptor PD-1. PD-L1 is widely expressed on various cell types, mainly in tumor cells, MDSCs, monocytes, macrophages, natural killer (NK) cells, dendritic cells (DCs), and activated T cells and also on immune-privileged sites such as the brain, cornea, and retina. In normal physiological conditions, the activation of the PD-1/PD-L1 signaling pathway is closely related to the induction and maintenance of peripheral tolerance, maintenance of T cell immune homeostasis, avoiding, hyperactivation and protecting against immune-mediated tissue damage. In disease states, PD-L1 interacts with its receptor programmed death 1 (PD-1), transmitting a negative signal to control a series of processes of T cell-mediated cellular immune responses, including priming, growth, proliferation and apoptosis, and functional maturation, leading to immune escape.

Immune checkpoint inhibitors (ICIs) have changed the treatment landscape of many tumors, inducing durable responses in some cases, Tumor mutational load, CD8⁺ T cell density and PD-L1 expression have each been proposed as distinct biomarkers of response to PD-1/-L1 antagonists. One of the main challenges for immune checkpoint blockade antibodies lies in malignancies with limited T-cell responses or immunologically “cold” tumors. These cold tumors contain few infiltrating T cells and are not recognized and do not provoke a strong response by the immune system, making them difficult to treat with current immunotherapies. Present inventors surprisingly found that GM-CSF upregulates PD-L1, contributing to immunosuppressive activity. Converting a “cold” tumor to a “hot” tumor is one of the milestones in cancer treatment.

### GM-CSF Antagonists

### GM-CSF Signaling

GM-CSF is a type I proinflammatory cytokine which enhances survival and proliferation of a broad range of hematopoietic cell types. It is a growth factor first identified as an inducer of differentiation and proliferation of myeloid cells (e.g., neutrophils, basophils, eosinophils, monocytes, and macrophages) (Wicks I P and Roberts A W. Nat Rev Rheumatol. 2016, 12 (1):37-48). Studies using different approaches have demonstrated that with GM-CSF overexpression, pathological changes almost always follow (Hamilton J A et al., Growth Factors. 2004, 22 (4):225-31). GM-CSF enhances trafficking of myeloid cells through activated endothelium of blood vessels and can also contribute to monocyte and macrophage accumulation in blood vessels during inflammation. GM-CSF also promotes activation, differentiation, survival, and proliferation of monocytes and macrophages as well as resident tissue macrophages in inflamed tissues. It regulates the phenotype of antigen-presenting cells in inflamed tissues by promoting the differentiation of infiltrating monocytes into M1 macrophages and monocyte-derived dendritic cells (MoDCs). Moreover, the production of IL-23 by macrophages and MoDCs, in combination with other cytokines such as IL-6 and IL-1, modulates T-cell differentiation.

Together with M-CSF (macrophage-colony stimulating factor), GM-CSF regulates the number and function of macrophages. Macrophages activated by GM-CSF acquire a series of effector functions, all of which identify them as inflammatory macrophages. GM-CSF-activated macrophages produce proinflammatory cytokines, including TNF, IL-1β, IL-6, IL-23 and IL-12 and chemokines, such as CCLS, CCL22, and CCL24, which recruit T cells and other inflammatory cells into the tissue microenvironment.

The GM-CSF receptor is a member of the haematopoietin receptor superfamily. It is heterodimeric, consisting of an alpha and a beta subunit. The alpha subunit is highly specific for GM-CSF, whereas the beta subunit is shared with other cytokine receptors, including IL-3 and IL-5. This is reflected in a broader tissue distribution of the beta receptor subunit. The alpha subunit, GM-CSFRα, is primarily expressed on myeloid cells and non-hematopoietic cells, such as neutrophils, macrophages, eosinophils, dendritic cells, endothelial cells and respiratory epithelial cells. Full length GM-CSFRα is a 400 amino acid type I membrane glycoprotein that belongs to the type I cytokine receptor family and consists of a 22 amino acid signal peptide (positions 1-22), a 298 amino acid extracellular domain (positions 23-320), a transmembrane domain from positions 321-345 and a short 55 amino acid intra-cellular domain. The signal peptide is cleaved to provide the mature form of GM-CSFRα as a 378 amino acid protein. Complementary DNA (cDNA) clones of the human and murine GM-CSFRα are available and, at the protein level, the receptor subunits have 36% identity. GM-CSF is able to bind with relatively low affinity to the a subunit alone (Kd 1-5 nM) but not at all to the β subunit alone. However, the presence of both α and β subunits results in a high affinity ligand-receptor complex (Kd˜100 pM). GM-CSF signaling occurs through its initial binding to the GM-CSFRα chain and then cross-linking with a larger subunit the common β chain to generate the high affinity interaction, which phosphorylates the JAK-STAT pathway. This interaction is also capable of signaling through tyrosine phosphorylation and activation of the MAP kinase pathway.

Pathologically, GM-CSF has been shown to play a role in exacerbating inflammatory, respiratory and autoimmune diseases. Neutralization of GM-CSF binding to GM-CSFRα is therefore a therapeutic approach to treating diseases and conditions mediated through GM-CSFR. Accordingly, the invention relates to a binding member that binds human GM-CSF or GM-CSFRα, or inhibits the binding of human GM-CSF to GM-CSFRα, and/or inhibits signaling that results from GM-CSF ligand binding to the receptor. Upon ligand binding, GM-CSFR triggers stimulation of multiple downstream signaling pathways, including JAK2/STAT5, the MAPK pathway, and the PI3K pathway; all relevant in activation and differentiation of myeloid cells. The binding member may be a reversible inhibitor of GM-CSF signaling through the GM-CSFR.

### GM-CSF Antagonists

A GM-CSF antagonist suitable for the present invention includes those therapeutic agents that can reduce, inhibit or abolish one or more GM-CSF mediated signaling including those described herein. For example, a suitable GM-CSF antagonist according to the invention includes, but is not limited to an anti-GM-CSF antibody or a fragment thereof, a soluble GM-CSF receptor and variants thereof including fusion proteins such as a GM-CSF soluble receptor-Fc fusion protein, an anti-GM-CSF receptor antibody or a fragment thereof, to name but a few.

In some embodiments, a suitable GM-CSF antagonist is an anti-GM-CSFRα antibody. Exemplary anti-GM-CSFRα monoclonal antibodies include those described in the international application PCT/GB2007/001108 filed on Mar. 27, 2007 which published as WO2007/110631, the EP application 120770487 filed on Oct. 10, 2010, U.S. application Ser. No. 11/692,008 filed on Mar. 27, 2007, U.S. application Ser. No. 12/294,616 filed on Sep. 25, 2008, U.S. application Ser. No. 13/941,409 filed on Jul. 12, 2013, U.S. application Ser. No. 14/753,792 filed on Nov. 30, 2010, international application PCT/EP2012/070074 filed on Oct. 10, 2012, which published as WO/2013/053767, international application PCT/EP2015/060902 filed on May 18, 2015, which published as WO2015/177097, international application PCT/EP2017/062479, filed on May 23, 2017, each of which are hereby incorporated by reference in their entirety. In one embodiment, the anti-GM-CSFRα monoclonal antibody is mavrilimumab. WO2007/110631 reports the isolation and characterization of the anti-GM-CSFRα antibody mavrilimumab and variants of it, which share an ability to neutralize the biological activity of GM-CSFRα with high potency. The functional properties of these antibodies are believed to be attributable, at least in part, to binding a Tyr-Leu-Asp-Phe-Gln motif at positions 226 to 230 of human GM-CSFRα, thereby inhibiting the association between GM-CSFRα and its ligand GM-CSF. Mavrilimumab is a human IgG4 monoclonal antibody designed to modulate macrophage activation, differentiation and survival by targeting the GM-CSFRα. It is a potent neutralizer of the biological activity of GM-CSFRα and, was shown to exert therapeutic effects by binding GM-CSFRα on leukocytes within the synovial joints of RA patients, leading to reduced cell survival and activation. The safety profile of the GM-CSFRα antibody mavrilimumab for in vivo use to date has been established in a Phase II clinical trial for rheumatoid arthritis (RA).

In certain embodiments, the antibody is comprised of two light chains and two heavy chains. The heavy chain variable domain (VH) comprises an amino acid sequence identified in SEQ ID NO: 1. The light chain variable domain (VL) comprises an amino acid sequence identified in SEQ ID NO: 2. The heavy and light chains each comprise complementarity determining regions (CDRs) and framework regions in the following arrangement:

FR1-CDR1-FR2-CDR2-FR3-CDR3-FR4

The mavrilimumab antibody heavy chain comprises CDRs: HCDR1, HCDR2, HCDR3 as identified by the amino acid sequences in SEQ ID NO: 3, 4 and 5 respectively. The light chain comprises CDRs: LCDR1, LCDR2, LCDR3 as identified by the amino acid sequences in SEQ ID NO: 6, 7 and 8 respectively.

Anti-GM-CSFRα Heavy Chain Variable Domain Amino
Acid Sequence
(SEQ ID NO: 1)
QVQLVQSGAEVKKPGASVKVSCKVSGYTLTELSIHWVRQAPGKGLEWM
GGFDPEENEIVYAQRFQGRVTMTEDTSTDTAYMELSSLRSEDTAVYYCA
IVGSFSPLTLGLWGQGTMVTVSS
Anti-GM-CSFRα Light Chain Variable Domain Amino
Acid Sequence
(SEQ ID NO: 2)
QSVLTQPPSVSGAPGQRVTISCTGSGSNIGAPYDVSWYQQLPGTAPKLL
IYHNNKRPSGVPDRFSGSKSGTSASLAITGLQAEDEADYYCATVEAGLS
GSVFGGGTKLTVL
Anti-GM-CSFRα Heavy Chain Variable Domain CDR1
(HCDR1) Amino Acid Sequence
(SEQ ID NO: 3)
ELSIH
Anti-GM-CSFRα Heavy Chain Variable Domain CDR2
(HCDR2) Amino Acid Sequence
(SEQ ID NO: 4)
GFDPEENEIVYAQRFQG
Anti-GM-CSFRα Heavy Chain Variable Domain CDR3
(HCDR3) Amino Acid Sequence
(SEQ ID NO: 5)
VGSFSPLTLGL
Anti-GM-CSFRα Light Chain Variable Domain CDR 1
(LCDR1 Amino Acid Sequence
(SEQ ID NO: 6)
TGSGSNIGAPYDVS
Anti-GM-CSFRα Light Chain Variable Domain CDR 2
(LCDR2) Amino Acid Sequence
(SEQ ID NO: 7)
HNNKRPS
Anti-GM-CSFRα Light Chain Variable Domain CDR3
(LCDR3) Amino Acid Sequence
(SEQ ID NO: 8)
ATVEAGLSGSV

In some embodiments the anti-GM-CSFRα antibody for cancer treatment is a variant of mavrilimumab, selected from the GM-CSFα binding members disclosed in the application WO2007/11063 and WO2013053767, which, together with the sequences disclosed therein, are incorporated by reference in its entirety.

In some embodiments the anti-GM-CSFRα antibody for cancer treatment comprises CDR amino acid sequences with at least 75%, 80%, 85%, 90%, 91%, 92%, 93%, 94%, 95%, 96%, 97%, 98%, 99% or more identity with one or more of SEQ ID NO:3, SEQ ID NO: 4, SEQ ID NO: 5, SEQ ID NO: 6, SEQ ID NO: 7, and SEQ ID NO: 8.

In some embodiments the anti-GM-CSFRα antibody comprises a light chain variable domain having an amino acid sequence at least 90% identical to SEQ ID NO: 2 and a heavy chain variable domain having an amino acid sequence at least 90% identical to SEQ ID NO: 1. In some embodiments of the invention, an anti-GM-CSFRα antibody has a light chain variable domain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 2 and a heavy chain variable domain amino acid sequence with at least 50%, 55%, 60%, 65%, 70%, 75%, 80%, 85%, 90%, 91%, 92%, 93%, 94%, 95%, 96%, 97%, 98%, 99% or more identity to SEQ ID NO: 1. In some embodiments of the invention, an anti-GM-CSFRα antibody comprises a light chain variable domain that has the amino acid sequence set forth in SEQ ID NO: 2 and a heavy chain variable domain that has the amino acid sequence set forth in SEQ ID NO: 1. In some embodiments of the invention, a heavy chain constant region of an anti-GM-CSFRα antibody comprises CH1, hinge and CH2 domains derived from an IgG4 antibody fused to a CH3 domain derived from an IgG1 antibody. In some embodiments of the invention, a heavy chain constant region of an anti-GM-CSFRα antibody is, or is derived from, an IgG1, IgG2 or IgG4 heavy chain constant region. In some embodiments of the invention, a light chain constant region of an anti-GM-CSFRα antibody is, or is derived from, a lambda or kappa light chain constant region.

In some embodiments, the anti-GM-CSFRα inhibitor is a fragment of mavrilimumab antibody. In some embodiments the inhibitor comprises a single chain variable fragment (ScFv) comprising at least any one of the CDR sequences of SEQ ID NO: 3, 4, 5, 6, 7, or 8. In some embodiments the inhibitor is a fusion molecule comprising at least any one of the CDR sequences of SEQ ID NO: 3, 4, 5, 6, 7, or 8. In some embodiments, the anti-GM-CSFRα inhibitor sequence is a bispecific antibody comprising at least one of the CDR sequences of SEQ ID NO: 3, 4, 5, 6, 7, or 8.

In other embodiments, a suitable GM-CSF antagonist is an anti-GM-CSF antibody. Exemplary anti-GM-CSF monoclonal antibodies include those described in the international application PCT/EP2006/004696 filed on May 17, 2006 which published as WO2006/122797, international application PCT/EP2016/076225 filed on Oct. 31, 2016, which published as WO2017/076804, and international application PCT/US2018/053933 filed on Oct. 2, 2018, which published as WO/2019/070680 each of which, including the sequences disclosed therein, are hereby incorporated by reference in their entirety. In one embodiment, the anti-GM-CSF monoclonal antibody is otilimab. In one embodiment, the anti-GM-CSF monoclonal antibody is namilumab. In one embodiment, the anti-GM-CSF monoclonal antibody is lenzilumab. In one embodiment, the anti-GM-CSF monoclonal antibody is gimsilumab. In one embodiment, the anti-GM-CSF monoclonal antibody is TJM2.

An anti-GM-CSFRα or anti-GM-CSF antibody of the present disclosure may be multispecific, e.g., bispecific. An antibody of the may be mammalian (e.g., human or mouse), humanized, chimeric, recombinant, synthetically produced, or naturally isolated. Exemplary antibodies of the present disclosure include, without limitation, IgG (e.g., IgG1, IgG2, IgG3, and IgG4), IgM, IgA (e.g., IgA1, IgA2, and IgAsec), IgD, IgE, Fab, Fab′, Fab′2, F(ab′)2, Fd, Fv, Feb, scFv, scFv-Fc, and SMIP binding moieties. In certain embodiments, the antibody is an scFv. The scFv may include, for example, a flexible linker allowing the scFv to orient in different directions to enable antigen binding. In various embodiments, the antibody may be a cytosol-stable scFv or intrabody that retains its structure and function in the reducing environment inside a cell (see, e.g., Fisher and DeLisa, J. Mol. Biol. 385 (1): 299-311, 2009; incorporated by reference herein). In particular embodiments, the scFv is converted to an IgG or a chimeric antigen receptor according to methods known in the art. In embodiments, the antibody binds to both denatured and native protein targets. In embodiments, the antibody binds to either denatured or native protein.

In most mammals, including humans, whole antibodies have at least two heavy (H) chains and two light (L) chains connected by disulfide bonds. Each heavy chain consists of a heavy chain variable region (VH) and a heavy chain constant region (CH). The heavy chain constant region consists of three domains (CH1, CH2, and CH3) and a hinge region between CH1 and CH2. Each light chain consists of a light chain variable region (VL) and a light chain constant region (CL). The light chain constant region consists of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen.

Antibodies include all known forms of antibodies and other protein scaffolds with antibody-like properties. For example, the anti-GM-CSFRα antibody can be a monoclonal antibody, a polyclonal antibody, human antibody, a humanized antibody, a bispecific antibody, a monovalent antibody, a chimeric antibody, or a protein scaffold with antibody-like properties, such as fibronectin or ankyrin repeats. The antibody can have any of the following isotypes: IgG (e.g., IgG1, IgG2, IgG3, and IgG4), IgM, IgA (e.g., IgA1, IgA2, and IgAsec), IgD, or IgE.

An antibody fragment may include one or more segments derived from an antibody. A segment derived from an antibody may retain the ability to specifically bind to a particular antigen. An antibody fragment may be, e.g., a Fab, Fab′, Fab′2, F(ab′)2, Fd, Fv, Feb, scFv, or SMIP. An antibody fragment may be, e.g., a diabody, triabody, affibody, nanobody, aptamer, domain antibody, linear antibody, single-chain antibody, or any of a variety of multispecific antibodies that may be formed from antibody fragments.

Examples of antibody fragments include: (i) a Fab fragment: a monovalent fragment consisting of VL, VH, CL, and CH1 domains; (ii) a F(ab′)2 fragment: a bivalent fragment comprising two Fab fragments linked by a disulfide bridge at the hinge region; (iii) a Fd fragment: a fragment consisting of VH and CH1 domains; (iv) an Fv fragment: a fragment consisting of the VL and VH domains of a single arm of an antibody; (v) a dAb fragment: a fragment including VH and VL domains; (vi) a dAb fragment: a fragment that is a VH domain; (vii) a dAb fragment: a fragment that is a VL domain; (viii) an isolated complementarity determining region (CDR); and (ix) a combination of two or more isolated CDRs which may optionally be joined by one or more synthetic linkers. Furthermore, although the two domains of the Fv fragment, VL and VH, are coded for by separate genes, they can be joined, using recombinant methods, e.g., by a synthetic linker that enables them to be expressed as a single protein, of which the VL and VH regions pair to form a monovalent binding moiety (known as a single chain Fv (scFv)). Antibody fragments may be obtained using conventional techniques known to those of skill in the art, and may, in some instances, be used in the same manner as intact antibodies. Antigen-binding fragments may be produced by recombinant DNA techniques or by enzymatic or chemical cleavage of intact immunoglobulins. An antibody fragment may further include any of the antibody fragments described above with the addition of additional C-terminal amino acids, N-terminal amino acids, or amino acids separating individual fragments.

An antibody may be referred to as chimeric if it includes one or more antigen-determining regions or constant regions derived from a first species and one or more antigen-determining regions or constant regions derived from a second species. Chimeric antibodies may be constructed, e.g., by genetic engineering. A chimeric antibody may include immunoglobulin gene segments belonging to different species (e.g., from a mouse and a human).

An antibody may be a human antibody. A human antibody refers to a binding moiety having variable regions in which both the framework and CDR regions are derived from human immunoglobulin sequences. Furthermore, if the antibody contains a constant region, the constant region also is derived from a human immunoglobulin sequence. A human antibody may include amino acid residues not identified in a human immunoglobulin sequence, such as one or more sequence variations, e.g., mutations. A variation or additional amino acid may be introduced, e.g., by human manipulation. A human antibody of the present disclosure is not chimeric.

An antibody may be humanized, meaning that an antibody that includes one or more antigen-determining regions (e.g., at least one CDR) substantially derived from a non-human immunoglobulin or antibody is manipulated to include at least one immunoglobulin domain substantially derived from a human immunoglobulin or antibody. An antibody may be humanized using the conversion methods described herein, for example, by inserting antigen-recognition sequences from a non-human antibody encoded by a first vector into a human framework encoded by a second vector. For example, the first vector may include a polynucleotide encoding the non-human antibody (or a fragment thereof) and a site-specific recombination motif, while the second vector may include a polynucleotide encoding a human framework and a site-specific recombination complementary to a site-specific recombination motif on the first vector. The site-specific recombination motifs may be positioned on each vector such that a recombination event results in the insertion of one or more antigen-determining regions from the non-human antibody into the human framework, thereby forming a polynucleotide encoding a humanized antibody.

In certain embodiments, an antibody is converted from scFv to an IgG (e.g., IgG1, IgG2, IgG3, and IgG4). There are various methods in the art for converting scFv fragments to IgG. One such method of converting scFv fragments to IgG is disclosed in US patent application publication number 20160362476, the contents of which are incorporated herein by reference.

### Combination Therapy

### Immune Checkpoint Inhibitors (ICIs)

In some embodiments, the method of treating cancer according to the present invention comprises administering to a subject in need thereof a GM-CSF antagonist in combination with ICI.

In some embodiments, the ICI is a biologic therapeutic or a small molecule. In some embodiments, the ICI is a monoclonal antibody, a humanized antibody, a fully human antibody, a fusion protein or a combination thereof.

In some embodiments, the ICI inhibits a checkpoint protein which may be CTLA-4, PD-L1, PD-L2, PD-1, B7-H3, B7-H4, BTLA, HVEM, TIM-3, GAL-9, LAG3, VISTA, KIR, 2B4, CD160, CGEN-15049, CHK1, CHK2, A2aR, B-7 family ligands or a combination thereof. In some embodiments, the ICI interacts with a ligand of a checkpoint protein which may be CTLA-4, PD-L1, PD-L2, PD-1, B7-H3, B7-H4, BTLA, HVEM, TIM-3, GAL-9, LAG3, VISTA, KIR, 2B4, CD160, CGEN-15049, CHK 1, CHK2, A2aR, B-7 family ligands or a combination thereof.

In some embodiments, the ICI is an anti-CTLA-4 antibody. In some embodiments, the ICI is an anti-PD-L1 antibody. In some embodiments, the ICI is an anti-PD-L2 antibody. In some embodiments, the ICI is an anti-PD-1 antibody. In some embodiments, the ICI is an anti-B7-H3 antibody. In some embodiments, the ICI is an anti-B7-H4 antibody. In some embodiments, the ICI is an anti-BTLA antibody. In some embodiments, the ICI is an anti-HVEM antibody. In some embodiments, the ICI is an anti-TIM-3 antibody. In some embodiments, the ICI is an anti-GAL-9 antibody. In some embodiments, the ICI is an anti-LAG-3 antibody. In some embodiments, the ICI is an anti-VISTA antibody. In some embodiments, the ICI is an anti-MR antibody. In some embodiments, the ICI is an anti-2B4 antibody. In some embodiments, the ICI is an anti-CD160 antibody. In some embodiments, the ICI is an anti-CGEN-15049 antibody. In some embodiments, the ICI is an anti-CHK1 antibody. In some embodiments, the ICI is an anti-CHK2 antibody. In some embodiments, the ICI is an anti-A2aR antibody. In some embodiments, the checkpoint inhibitor is an anti-B-7 antibody.

In some embodiments, the PD-1 antibody is pembrolizumab. In some embodiments, the PD-1 antibody is nivolumab. In some embodiments, the PD-1 antibody is cemiplimab. In some embodiments, the PD-L1 antibody is atezolizumab. In some embodiments, the PD-L1 antibody is avelumab. In some embodiments, the PD-L1 antibody is durvalumab. In some embodiments, the CTLA-4 antibody is ipillimumab.

### Additional Therapeutic Agents

In some embodiments, the method of treating cancer according to the present invention comprises administering to a subject in need thereof a GM-CSF antagonist in combination with an additional therapeutic agent. In certain embodiments, the additional agent is a cancer therapy comprising chemotherapy and/or radiation therapy. In certain embodiments, the additional therapeutic agent comprises a recombinant protein or monoclonal antibody. In certain embodiments, the recombinant protein or monoclonal antibody comprises Etaracizumab (Abegrin), Tacatuzumab tetraxetan, Bevacizumab (Avastin), Labetuzumab, Cetuximab (Erbitux), Obinutuzumab (Gazyva), Trastuzumab (Herceptin), Clivatuzumab, Trastuzumab emtansine (Kadcyla), Ramucirumab, Rituximab (MabThera, Rituxan), Gemtuzumab ozogamicin (Mylotarg), Pertuzumab (Omnitarg), Girentuximab (Rencarex), or Nimotuzumab (Theracim, Theraloc).

In certain embodiments, the GM-CSF antagonist comprises an immunomodulator that targets a checkpoint inhibitor as described in the Checkpoint Inhibitors section above. In certain embodiments, the immunomodulator comprises Nivolumab, Ipilimumab, Atezolizumab, or Pembrolizumab. In certain embodiments, the additional therapeutic agent is a chemotherapeutic agent. In certain embodiments, the chemotherapeutic agent is an alkylating agent (e.g., cyclophosphamide, ifosfamide, chlorambucil, busulfan, melphalan, mechlorethamine, uramustine, thiotepa, nitrosoureas, or temozolomide), an anthracycline (e.g., doxorubicin, adriamycin, daunorubicin, epirubicin, or mitoxantrone), a cytoskeletal disruptor (e.g., paclitaxel or docetaxel), a histone deacetylase inhibitor (e.g., vorinostat or romidepsin), an inhibitor of topoisomerase (e.g., irinotecan, topotecan, amsacrine, etoposide, or teniposide), a kinase inhibitor (e.g., bortezomib, erlotinib, gefitinib, imatinib, vemurafenib, or vismodegib), a nucleoside analog or precursor analog (e.g., azacitidine, azathioprine, capecitabine, cytarabine, fluorouracil, gemcitabine, hydroxyurea, mercaptopurine, methotrexate, or thioguanine), a peptide antibiotic (e.g., actinomycin or bleomycin), a platinum-based agent (e.g, cisplatin, oxaloplatin, or carboplatin), or a plant alkaloid (e.g., vincristine, vinblastine, vinorelbine, vindesine, podophyllotoxin, paclitaxel, or docetaxel). In some embodiments, the chemotherapeutic agent is a nucleoside analog. In some embodiments, the chemotherapeutic agent is gemcitabine. In certain embodiments, the additional therapeutic agent is radiation therapy.

### Treatment of Cancer

The present invention may be used to treat various cancers, for example, those immune checkpoint inhibitory (ICI) refractory or resistant cancers, or late stage or metastatic cancers.

In some embodiments, the cancer is any solid tumor or liquid cancers, including urogenital cancers (such as prostate cancer, renal cell cancers, bladder cancers), gynecological cancers (such as ovarian cancers, cervical cancers, endometrial cancers), lung cancer, gastrointestinal cancers (such as non-metastatic or metastatic colorectal cancers, pancreatic cancer, gastric cancer, oesophageal cancers, hepatocellular cancers, cholangiocellular cancers), head and neck cancer (e.g. head and neck squamous cell cancer), brain cancers including malignant gliomas and brain metastases, malignant mesothelioma, non-metastatic or metastatic breast cancer (e.g. hormone refractory metastatic breast cancer), malignant melanoma, Merkel Cell Carcinoma or bone and soft tissue sarcomas, and haematologic neoplasias, such as multiple myeloma, acute myelogenous leukemia (AML), chronic myelogenous leukemia (CML), chronic myelomonocytic leukemia (CMML), juvenile myelomonocytic leukemia (JMML), myelodysplastic syndrome and acute lymphoblastic leukemia. In a some embodiment, the disease is non-small cell lung cancer (NSCLC), breast cancer (e.g. stage IV breast cancer, hormone refractory metastatic breast cancer), head and neck cancer (e.g. head and neck squamous cell cancer), metastatic colorectal cancers, hormone sensitive or hormone refractory prostate cancer, colorectal cancer (e.g. stage IV colorectal cancer), ovarian cancer, hepatocellular cancer, renal cell cancer, soft tissue sarcoma, or small cell lung cancer.

As used herein, the term “cancer” refers to the broad class of disorders characterized by hyperproliferative cell growth, either in vitro (e.g., transformed cells) or in vivo. Conditions which can be treated or prevented by the compositions and methods of the invention include, e.g., a variety of neoplasms, including benign or malignant tumors, a variety of hyperplasias, or the like. Compounds and methods of the invention can achieve the inhibition and/or reversion of undesired hyperproliferative cell growth involved in such conditions.

Examples of cancer include Acute Lymphoblastic Leukemia, Adult; Acute Lymphoblastic Leukemia, Childhood; Acute Myeloid Leukemia, Adult; Adrenocortical Carcinoma; Adrenocortical Carcinoma, Childhood; AIDS-Related Lymphoma; AIDS-Related Malignancies; Anal Cancer; Astrocytoma, Childhood Cerebellar; Astrocytoma, Childhood Cerebral; Bile Duct Cancer, Extrahepatic; Bladder Cancer; Bladder Cancer, Childhood; Bone Cancer, Osteosarcoma/Malignant Fibrous Histiocytoma; Brain Stem Glioma, Childhood; Brain Tumor, Adult; Brain Tumor, Brain Stem Glioma, Childhood; Brain Tumor, Cerebellar Astrocytoma, Childhood; Brain Tumor, Cerebral Astrocytoma/Malignant Glioma, Childhood; Brain Tumor, Ependymoma, Childhood; Brain Tumor, Medulloblastoma, Childhood; Brain Tumor, Supratentorial Primitive Neuroectodermal Tumors, Childhood; Brain Tumor, Visual Pathway and Hypothalamic Glioma, Childhood; Brain Tumor, Childhood (Other); Breast Cancer; Breast Cancer and Pregnancy; Breast Cancer, Childhood; Breast Cancer, Male; Bronchial Adenomas/Carcinoids, Childhood: Carcinoid Tumor, Childhood; Carcinoid Tumor, Gastrointestinal; Carcinoma, Adrenocortical; Carcinoma, Islet Cell; Carcinoma of Unknown Primary; Central Nervous System Lymphoma, Primary; Cerebellar Astrocytoma, Childhood; Cerebral Astrocytoma/Malignant Glioma, Childhood; Cervical Cancer; Childhood Cancers; Chronic Lymphocytic Leukemia; Chronic Myelogenous Leukemia, Chronic Myelomonocytic Leukemia (CMML), Juvenile Myelomonocytic Leukemia (JMML); Chronic Myeloproliferative Disorders; Clear Cell Sarcoma of Tendon Sheaths; Colon Cancer; Colorectal Cancer, Childhood; Cutaneous T-Cell Lymphoma; Endometrial Cancer; Ependymoma, Childhood; Epithelial Cancer, Ovarian; Esophageal Cancer; Esophageal Cancer, Childhood; Ewing's Family of Tumors; Extracranial Germ Cell Tumor, Childhood; Extragonadal Germ Cell Tumor; Extrahepatic Bile Duct Cancer; Eye Cancer, Intraocular Melanoma; Eye Cancer, Retinoblastoma; Gallbladder Cancer; Gastric (Stomach) Cancer; Gastric (Stomach) Cancer, Childhood; Gastrointestinal Carcinoid Tumor; Germ Cell Tumor, Extracranial, Childhood; Germ Cell Tumor, Extragonadal; Germ Cell Tumor, Ovarian; Gestational Trophoblastic Tumor; Glioma. Childhood Brain Stem; Glioma. Childhood Visual Pathway and Hypothalamic; Hairy Cell Leukemia; Head and Neck Cancer; Hepatocellular (Liver) Cancer, Adult (Primary); Hepatocellular (Liver) Cancer, Childhood (Primary); Hodgkin's Lymphoma, Adult; Hodgkin's Lymphoma, Childhood; Hodgkin's Lymphoma During Pregnancy; Hypopharyngeal Cancer; Hypothalamic and Visual Pathway Glioma, Childhood; Intraocular Melanoma; Islet Cell Carcinoma (Endocrine Pancreas); Kaposi's Sarcoma; Kidney Cancer; Laryngeal Cancer; Laryngeal Cancer, Childhood; Leukemia, Acute Lymphoblastic, Adult; Leukemia, Acute Lymphoblastic, Childhood; Leukemia, Acute Myeloid, Adult; Leukemia, Acute Myeloid, Childhood; Leukemia, Chronic Lymphocytic; Leukemia, Chronic Myelogenous; Leukemia, Hairy Cell; Lip and Oral Cavity Cancer; Liver Cancer, Adult (Primary); Liver Cancer, Childhood (Primary); Lung Cancer, Non-Small Cell; Lung Cancer, Small Cell; Lymphoblastic Leukemia, Adult Acute; Lymphoblastic Leukemia, Childhood Acute; Lymphocytic Leukemia, Chronic; Lymphoma, AIDS-Related; Lymphoma, Central Nervous System (Primary); Lymphoma, Cutaneous T-Cell; Lymphoma, Hodgkin's, Adult; Lymphoma, Hodgkin's; Childhood; Lymphoma, Hodgkin's During Pregnancy; Lymphoma, Non-Hodgkin's, Adult; Lymphoma, Non-Hodgkin's, Childhood; Lymphoma, Non-Hodgkin's During Pregnancy; Lymphoma, Primary Central Nervous System; Macroglobulinemia, Waldenstrom's; Male Breast Cancer; Malignant Mesothelioma, Adult; Malignant Mesothelioma, Childhood; Malignant Thymoma; Medulloblastoma, Childhood; Melanoma; Melanoma, Intraocular; Merkel Cell Carcinoma; Mesothelioma, Malignant; Metastatic Squamous Neck Cancer with Occult Primary; Multiple Endocrine Neoplasia Syndrome, Childhood; Multiple Myeloma/Plasma Cell Neoplasm; Mycosis Fungoides; Myelodysplastic Syndromes; Myelogenous Leukemia, Chronic; Myeloid Leukemia, Childhood Acute; Myeloma, Multiple; Myeloproliferative Disorders, Chronic; Nasal Cavity and Paranasal Sinus Cancer; Nasopharyngeal Cancer; Nasopharyngeal Cancer, Childhood; Neuroblastoma; Neurofibroma; Non-Hodgkin's Lymphoma, Adult; Non-Hodgkin's Lymphoma, Childhood; Non-Hodgkin's Lymphoma During Pregnancy; Non-Small Cell Lung Cancer; Oral Cancer, Childhood; Oral Cavity and Lip Cancer; Oropharyngeal Cancer; Osteosarcoma/Malignant Fibrous Histiocytoma of Bone; Ovarian Cancer, Childhood; Ovarian Epithelial Cancer; Ovarian Germ Cell Tumor; Ovarian Low Malignant Potential Tumor; Pancreatic Cancer; Pancreatic Cancer, Childhood', Pancreatic Cancer, Islet Cell; Paranasal Sinus and Nasal Cavity Cancer; Parathyroid Cancer; Penile Cancer; Pheochromocytoma; Pineal and Supratentorial Primitive Neuroectodermal Tumors, Childhood; Pituitary Tumor; Plasma Cell Neoplasm/Multiple Myeloma; Pleuropulmonary Blastoma; Pregnancy and Breast Cancer; Pregnancy and Hodgkin's Lymphoma; Pregnancy and Non-Hodgkin's Lymphoma; Primary Central Nervous System Lymphoma; Primary Liver Cancer, Adult; Primary Liver Cancer, Childhood; Prostate Cancer; Rectal Cancer; Renal Cell (Kidney) Cancer; Renal Cell Cancer, Childhood; Renal Pelvis and Ureter, Transitional Cell Cancer; Retinoblastoma; Rhabdomyosarcoma, Childhood; Salivary Gland Cancer; Salivary Gland' Cancer, Childhood; Sarcoma, Ewing's Family of Tumors; Sarcoma, Kaposi's; Sarcoma (Osteosarcoma)/Malignant Fibrous Histiocytoma of Bone; Sarcoma, Rhabdomyosarcoma, Childhood; Sarcoma, Soft Tissue, Adult; Sarcoma, Soft Tissue, Childhood; Sezary Syndrome; Skin Cancer; Skin Cancer, Childhood; Skin Cancer (Melanoma); Skin Carcinoma, Merkel Cell; Small Cell Lung Cancer; Small Intestine Cancer; Soft Tissue Sarcoma, Adult; Soft Tissue Sarcoma, Childhood; Squamous Neck Cancer with Occult Primary, Metastatic; Stomach (Gastric) Cancer; Stomach (Gastric) Cancer, Childhood; Supratentorial Primitive Neuroectodermal Tumors, Childhood; T-Cell Lymphoma, Cutaneous; Testicular Cancer; Thymoma, Childhood; Thymoma, Malignant; Thyroid Cancer; Thyroid Cancer, Childhood; Transitional Cell Cancer of the Renal Pelvis and Ureter; Trophoblastic Tumor, Gestational; Unknown Primary Site, Cancer of, Childhood; Unusual Cancers of Childhood; Ureter and Renal Pelvis, Transitional Cell Cancer; Urethral Cancer; Uterine Sarcoma; Vaginal Cancer; Visual Pathway and Hypothalamic Glioma, Childhood; Vulvar Cancer; Waldenstrom's Macro globulinemia; and Wilms' Tumor.

Chronic myeloid leukemia (CML) affects the myeloid cells in the blood and bone marrow, while chronic myelomonocytic leukemia (CMML) and juvenile myelomonocytic leukemia (JMML) affect a specific myeloid cell called a monocyte, which helps to fight infections. For CMML, the median age at diagnosis ranges from 65 to 75 years. Common CMML symptoms include weakness, fatigue, unexplained bruising and/or bleeding, infection and enlarged liver and/or spleen. CMML is a myeloid neoplasm characterized by dysplasia, abnormal production and accumulation of monocytic cells and an elevated risk of transforming into acute leukemia. In some aspects, the present invention provides a method of treating CMML in a patient comprising administering to the patient in need of treatment a therapeutically effective dose of a GM-CSF antagonist, wherein the administration improves, stabilizes reduces one or more symptoms of CMML as compared to a control. The 2016 iteration of the World Health Organization (WHO) classification of myeloid neoplasms defines chronic myelomonocytic leukemia (CMML) as a clonal hematopoietic stem cell disorder characterized by the presence of sustained (>3 months) peripheral blood (PB) monocytosis (≥1×10⁹/L; monocytes≥10% of white blood cell count) along with dysplastic features in the bone marrow (BM). Secondary to the overlapping features of both, myelodysplastic syndromes (MDS) and myeloproliferative neoplasms (MPN), the classification of CMML as a unique myeloid neoplasm has undergone several changes dating back to the original French-American-British (FAB) co-operative group effort in 1982. Due to renewed evidence demonstrating clinical, morphological and molecular differences, the 2016 WHO classification has once again recommended categorization of CMML into “proliferative” (MPN-CMML) and “dysplastic” (MDS-CMML) sub-types; based on a white blood cell count of ≥13×10⁹/L for MPN-CMML and a white blood cell count of <13×10⁹/L for MDS-CMML. In some embodiments, a GM-CSF antagonist is administered to a patient with CMML, wherein the CMML is categorized as MPN-CMML. In some embodiments, a GM-CSF antagonist is administered to a patient with CMML, wherein the CMML is categorized as MDS-CMML. In addition, based on PB and BM blast %, CMML can be sub-classified into three categories; (a) CMML-0 (<2% PB blasts including promonocytes and <5% BM blasts), (b) CMML-1 (2-4% PB blasts including promonocytes and 5%-9% BM blasts), and (c) CMML-2 (>5% PB blasts including promonocytes and 10%-19% BM blasts and/or when any Auer rods are present). Mortality of CMML is ultimately caused by: 1) bone marrow failure; 2) break-through opportunistic infection (majority of patients); 3) transformation to AML; and/or 4) thrombosis/clot. In some embodiments, a GM-CSF antagonist is administered to a patient with CMML, wherein the CMML is classified as CMML-0. In some embodiments, a GM-CSF antagonist is administered to a patient with CMML, wherein the CMML is classified as CMML-1. In some embodiments, a GM-CSF antagonist is administered to a patient with CMML, wherein the CMML is classified as CMML-2.

CMML frequently progresses to acute myeloid leukemia (AML). Fifteen to thirty percent of patients will progress to AML, at which point survival rates drop to 4.7 months without a hematopoietic cell transplantation (HCT) and 14.3 months with an HCT. One known prognostic factor for survival is CMML subtype. The dysplastic and proliferative subtypes affect patients very differently: Patients with the dysplastic subtype have low blood counts and their natural history and clinical problems related to marrow failure are more similar to patients with MDS; those with the proliferative subtype have high blood counts and often have constitutional symptoms or symptoms related to organomegaly. Patients with proliferative forms of the disease also have shorter survival and a higher risk of transformation to AML. In some embodiments, administration of a GM-CSF antagonist to a patient with CMML reduces the time to transformation to AML, or prevents the transformation to AML.

JMML is most commonly diagnosed in infants and children younger than 6 years. Common JMML symptoms include pallor, developmental delays, decrease in appetite, irritability, enlarged abdomen, dry cough, rash, enlarged liver and/or spleen and enlarged lymph nodes. In some aspects, the present invention provides a method of treating JMML in a patient comprising administering to the patient in need of treatment a therapeutically effective dose of a GM-CSF antagonist, wherein the administration improves, stabilizes reduces one or more symptoms of JMML as compared to a control. In some embodiments, administration of a GM-CSF antagonist to a patient with JMML reduces the time to transformation to AML, or prevents the transformation to AML.

### Pharmaceutical Compositions and Administration

The antibodies or agents of the invention (also referred to herein as “active compounds”), and derivatives, fragments, analogs and homologs thereof, can be incorporated into pharmaceutical compositions suitable for administration. Such compositions typically comprise the antibody or agent and a pharmaceutically acceptable carrier. As used herein, the term “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, ringer's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e., topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e.g., with conventional suppository bases such as cocoa butter and other glycerides) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The pharmaceutical compositions can be included in a container, pack, or dispenser together with instructions for administration.

In some embodiments, the therapeutically effective dose of a GM-CSF antagonist is about 30 mg, 35 mg, 40 mg, 45 mg, 50 mg, 55 mg, 60 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 250 mg. In some embodiments, the therapeutically effective dose is about 30 mg, 35 mg, 40 mg, 45 mg, 50 mg, 55 mg, 60 mg, 70 mg, 275 mg, 280 mg, 285 mg, 290 mg, 295 mg, 300 mg, 325 mg, 350 mg, 375 mg, 400 mg, 425 mg, 450 mg, 475 mg, 500 mg, 550 mg, 650 mg, 625 mg, 650 mg, 675 mg, 700 mg, 725 mg, 750mg, or 800 mg.

In some embodiments, the therapeutically effective dose is about 225 mg. In some embodiments, the therapeutically effective dose is about 375 mg. In some embodiments, the therapeutically effective dose is about 450 mg. In some embodiments, the therapeutically effective dose is about 750 mg.

In some embodiments, the therapeutically effective dose of a GM-CSF antagonist is between 0.1 mg/kg and 15 mg/kg. In some embodiments, the therapeutically effective dose is between 2 mg/kg and 10 mg/kg. In some embodiments, the therapeutically effective dose is between 3 mg/kg and 10 mg/kg. In some embodiments, the therapeutically effective dose is between 5 mg/kg and 10 mg/kg. In some embodiments, the therapeutically effective dose is between 6 mg/kg and 10 mg/kg. In some embodiments, the therapeutically effective dose is about 1 mg/kg. In some embodiments, the therapeutically effective dose is about 2 mg/kg. In some embodiments, the therapeutically effective dose is about 3 mg/kg. In some embodiments, the therapeutically effective dose is about 4 mg/kg. In some embodiments, the therapeutically effective dose is about 5 mg/kg. In some embodiments, the therapeutically effective dose is about 6 mg/kg. In some embodiments, the therapeutically effective dose is about 7 mg/kg. In some embodiments, the therapeutically effective dose is about 8 mg/kg. In some embodiments, the therapeutically effective dose is about 9 mg/kg. In some embodiments, the therapeutically effective dose is about 10 mg/kg. In some embodiments, the therapeutically effective dose is about 11 mg/kg. In some embodiments, the therapeutically effective dose is about 12 mg/kg. In some embodiments, the therapeutically effective dose is about 15 mg/kg.

In some embodiments, a GM-CSF antagonist is administered at a particular administration interval. In some embodiments, the administration interval is once every week.

In some embodiments, the administration interval is at least five days.

In some embodiments, the administration interval is once every two weeks.

In some embodiments, the administration interval is once every three weeks.

In some embodiments, the administration interval is once every four weeks.

In some embodiments, the administration interval is once every five weeks.

In certain embodiments, the GM-CSF antagonist is administered by intravenous administration.

In some embodiments, the GM-CSF antagonist administered as an initial loading dose followed by at least one maintenance dose. In some embodiments, the maintenance dose is lower than the initial loading dose. In some embodiments, the maintenance dose is higher than the initial loading dose. In some embodiments, the maintenance dose is lower than the initial loading dose.

### EXAMPLES

The present invention is further illustrated, but not limited, by the slides accompanying this specification. While certain compounds, compositions and methods of the present invention have been described with specificity in accordance with certain embodiments, the following examples serve only to illustrate the invention and are not intended to limit the same.

### Example 1. An Anti-GM-CSFRα Antibody Rescues T Cell Proliferation

The study in this example illustrates that suppressive potential of myeloid populations on T cell proliferation can be rescued by GM-CSF antagonist.

T cell proliferation assay with CD14+ cells with or without treatment of anti-GM-CSFRα antibody was performed. Briefly, CD14+ (MDSC) cells were isolated from the blood samples (PBMCs) obtained from pancreatic cancer patients according to methods known in the art. The isolated CD14+ cells were treated with 100 μg/mL of anti-GM-CSFRα antibodies for 48 hours. Next, the CD3+ T-cells labeled with carboxy-fluorescein diacetate succinimidyl ester (CFSE) were co-cultured with anti-GM-CSFRα antibody-treated or untreated CD14+ cells for 96 hours and proliferation was determined by CFSE dilution (divided cells). T cells without co-culture were used as a negative control.

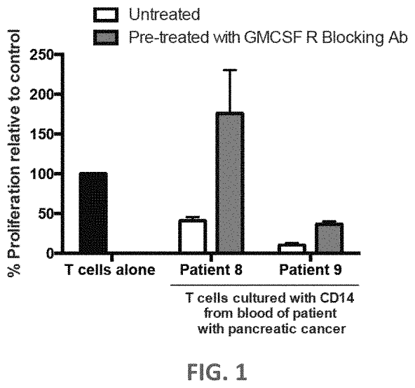

As shown in FIG. 1, T cell proliferation is suppressed significantly following culture with the untreated CD14+ cells. On the other hand, anti-GM-CSFRα antibody-treated CD14+ cells showed an increased T cell proliferation, suggesting that an addition of anti-GM-CSFRα antibody rescued T cell proliferation and prevented suppressive potential of MDSCs.

### Example 2. Cancer Cell Conditioned Medium Polarize Monocytes to Phenotypic MDSCs

In this study, various cancer cell lines were used to illustrate an increase in phenotypic MDSCs (HLA-DR^{low}) when CD+14 monocytes were incubated with conditioned medium from GM-CSF expressing cancer cells.

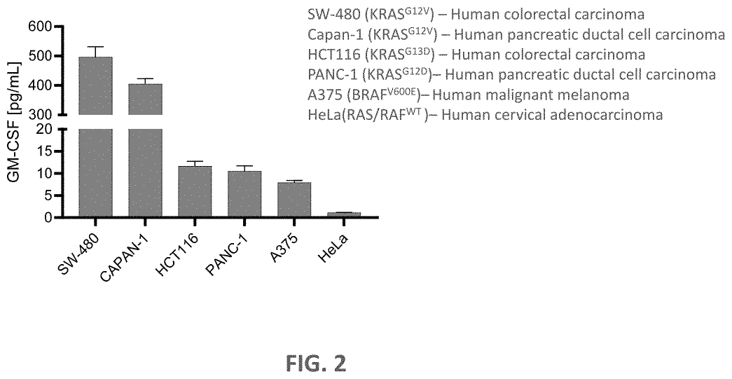

Cancer cell lines were analyzed for expression of GM-CSF. In this particular study, two colorectal carcinoma (HCT116 and SW-480), two pancreatic carcinoma (Panc-1 and Capan-1), cervical adenocarcinoma (HeLa), and malignant melanoma (A375) cell lines were measured for GM-CSF expression. Among the cell lines tested, SW-480, CAPAN-1, HCT116, PANC-1 have KRAS mutation A375 have RAF mutations, and HeLa are RAS/RAF wild type. As shown in FIG. 2, cancer cell lines express GM-CSF at different levels. In particular, SW480 and Capan-1, which are KRAS-mutant cancer cell lines, have relatively high expression of GM-CSF, whereas HeLa cells, which lack KRAS mutation, have relatively low expression of GM-CSF.

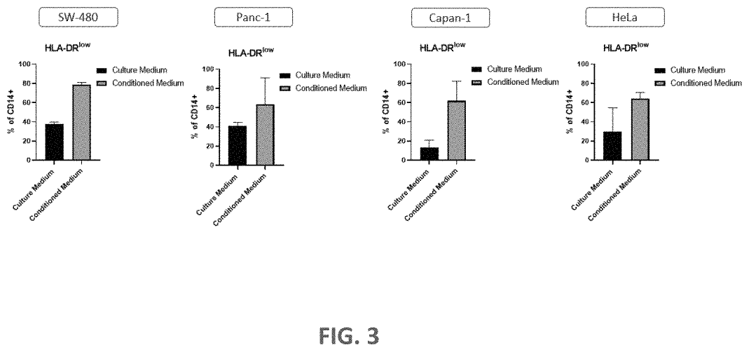

To generate tumor-conditioned media (CM), cell lines were plated and cultured according to methods known in the art. CD14+ cells were then cultured in the presence of CM for 6 days and analyzed for gene and protein expression. Low levels of HLA-DR biomarker is indicative of MDSC phenotype. FIG. 3 shows an increase in phenotypic MDSCs when CD14+ monocytes were incubated with conditioned medium from GM-CSF expressing cancer cells, as compared to CD+14 cells that were grown in normal culture medium. Results show that CM from cancer cell lines with high GM-CSF expression have high induction of MDSCs, suggesting that GM-CSF contributes to polarization of monocytes to phenotypic MDSCs.

### Example 3. An Anti-GM-CSFRα Antibody Blocks PD-L1 Upregulation in MDSCs

The study in this example illustrates a surprising finding by the present inventors that GM-CSF induces expression of PD-L1 on phenotypic MDSCs. Notably, treatment with a GM-CSF antagonist is sufficient to represses the expression of PD-L1 on monocytes treated with conditioned medium (CM) from GM-CSF expressing cancer cell lines.

One of the main challenges for immune checkpoint blockade antibodies lies in malignancies with limited T-cell responses or immunologically “cold” tumors. These cold tumors contain few infiltrating T cells and are not recognized and do not provoke a strong response by the immune system, making them difficult to treat with current immunotherapies. Present inventors surprisingly found that GM-CSF upregulates PD-L1, a checkpoint protein on the surface of MDSCs that contributes to immunosuppressive activity. The study in this example shows that an anti-GM-CSFRα antibody can be used to convert a “cold” tumor to a “hot” tumor, possibly increasing effectiveness and sensitivity of immunotherapy.

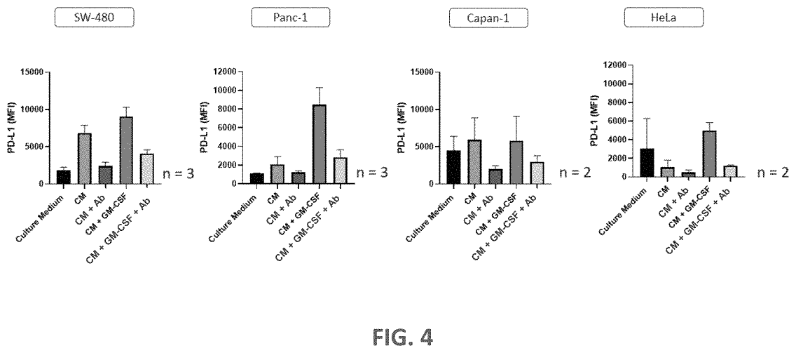

In this study, various cancer cell lines were used to evaluate changes in PD-L1 expression levels on MDSCs when CD14+ monocytes were incubated with conditioned medium from GM-CSF expressing cancer cells. As shown in FIG. 4, adding conditioned medium from GM-CSF expressing cancer cell lines to the CD14+ monocytes increased the level of PD-L1 expression as compared to the baseline (culture medium only). HeLa cell lines, which exhibited low levels of GM-CSF expression (see FIG. 2) did not upregulate PD-L1 expression. Adding recombinant GM-CSF in combination with CM (CM+GM-CSF) increased the expression of PD-L1, indicating that GM-CSF induces expression of PD-L1 on phenotypic MDSCs. The spike in PD-L1 was more pronounced in cell lines that had low baseline PD-L1 expression with CM only (e.g., Panc-1 and HeLa cells). The effect of an anti-GM-CSFRα antibody on PD-L1 expression was also examined. Treating the MDSCs with anti-GM-CSFRα antibody in CM, in the absence or in presence of recombinant GM-CSF (CM+Ab and CM+GM-CSF+Ab, respectively) resulted in a markedly decreased level of PD-L1 as compared to CM only or CM+GM-CSF, respectively. These data show that an anti-GM-CSFRα antibody suppresses PD-L1 upregulation in CD14+ monocytes (MDSCs) treated with condition medium conditioned by from GM-CSF expressing cancer cell lines.

### Example 4. An Anti-GM-CSFRα Antibody Represses PD-L1 Expression in MDSCs

The study in this example shows that a GM-CSF antagonist is able to suppress PD-L1 expression on MDSCs treated with condition medium from GM-CSF expressing cancer cell lines (CM), whether the GM-CSF antagonist is added concurrently with, or after the CM treatment when PD-L1 levels on the MDSCs are already increased.

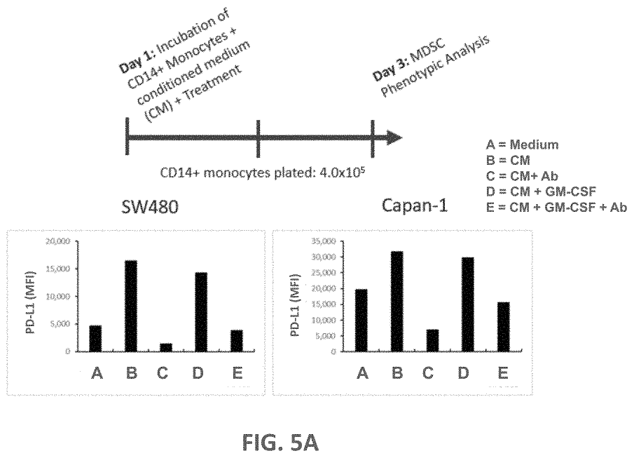

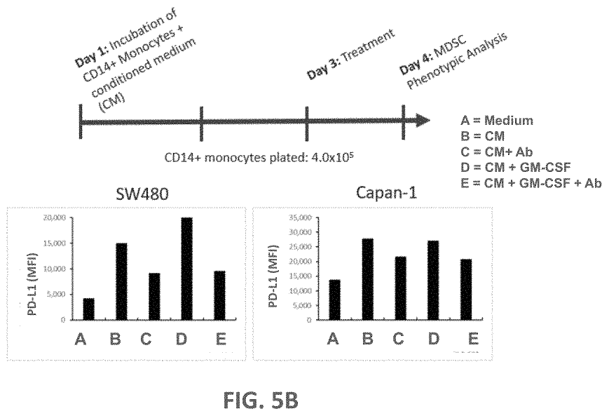

As shown in FIG. 5A, conditioned medium from GM-CSF cancer cell line (CM) with or without recombinant GM-CSF (at 10 ng/mL) and anti-GM-CSFRα antibody (at 100 μg/mL) (as shown in Table 1) were added to CD14+ monocytes (MDSCs) at day 1. After three days of incubation, the MDSC cells were analyzed for expression of PD-L1.

TABLE 1

| Sample | Content |
|---|---|
| A | Control (Culture medium) |
| B | CM |
| C | CM + anti-GM-CSFRα antibody |
| D | CM + GM-CSF |
| E | CM + GM-CSF + anti-GM-CSFRα |
| | antibody |

Consistent with example 3, adding conditioned medium from cancer cell lines expressing GM-CSF to the MDSCs (B; CM) or CM with recombinant GM-CSF (D; CM+GM-CSF) increased the level of PD-L1 expression as compared to the baseline (A; culture medium only). Moreover, when an anti-GM-CSFRα antibody was added concurrently with CM or CM+GM-CSF, as shown in samples C and E of FIG. 5A, respectively, a decrease in PD-L1 was observed, suggesting that the anti-GM-CSFRα antibody blocks upregulation of PD-L1 on MDSCs.

Next, the effect of adding an anti-GM-CSFRα antibody after incubation of MDSCs with conditioned medium to examine the effect of GM-CSF blockade on PD-L1 expression levels when elevated level of PD-L1 on MDSCs was already established. In this particular setup, MDSCs were cultured in conditioned medium with or without recombinant GM-CSF (samples B-E of FIG. 5B) for 48 hours. Then, an anti-GM-CSFRα antibody was added at day 3 to samples C and E of FIG. 5B (after 48 hours). MDSCs in sample A were incubated in culture medium for three days as a control. Phenotypic analysis of MDSCs was performed on day 4. As shown in FIG. 5B, treatment of MDSCs with an anti-GM-CSFRα antibody after MDSCs were cultured with conditioned medium from GM-CSF expressing cancer cell lines (samples C and E) repressed the expression level of PD-L1 on MDSCs. Notably, PD-L1 expression in sample C and E were decreased as compared to samples B and D, respectively, after just 24 hours of treatment with an anti-GM-CSFRα antibody.

### Example 5. An Anti-GM-CSFRα Antibody Reduces MDSC Mediated T-cell Suppression

The study in this example further illustrates that suppressive potential of myeloid populations on T cell proliferation can be repressed by a GM-CSF antagonist.

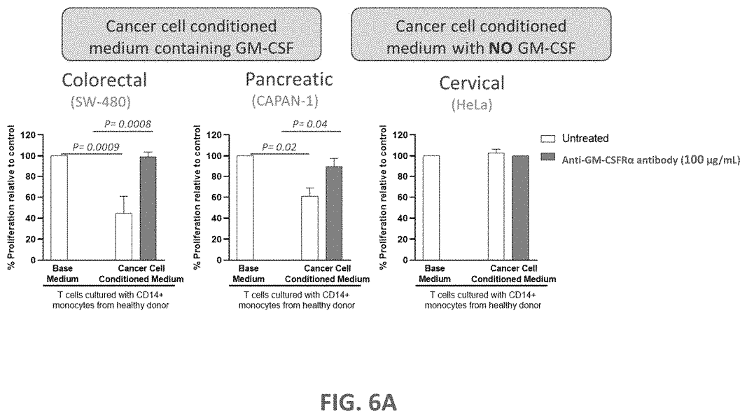

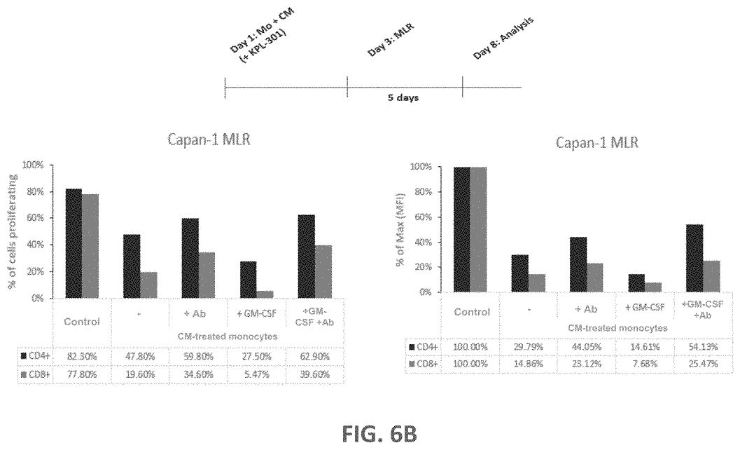

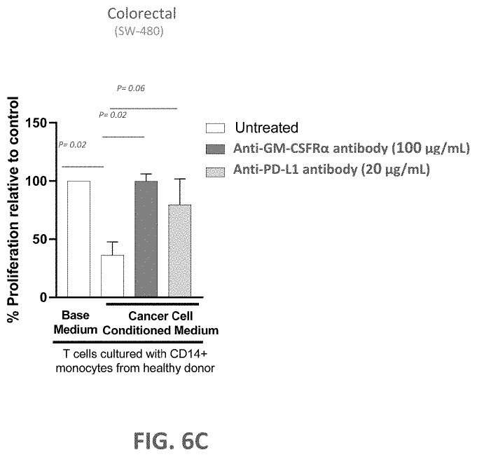

In this particular experiment, monocytes treated with conditioned medium from GM-CSF expressing cancer cell lines were used in T cell proliferation assay. Briefly, monocytes were cultured in conditioned medium from GM-CSF expressing cancer cell lines (CM) for three days (CM-treated monocytes). CD8+ T cells (1×10⁵ cells) were prepared by labeling with CFSE and stimulation with of IL-2 and soluble CD3/CD28 T cell activator in IMDM cell culture medium. On day 3, the stimulated T cells were co-cultured with CM-treated monocytes (at a ratio of 2:1 monocyte:T cell) with or without anti-GM-CSFRα antibody (100 μg/mL) in a mix lymphocyte reaction as shown in FIG. 6A. T-cells were expanded for 5 days and cell proliferation was evaluated by CFSE dilution in CD4+ and CD8+ T cells, which are markers for helper T and cytotoxic T cells, by flow cytometry. FIG. 6A shows that anti-GM-CSFRα antibody disrupted the ability of GM-CSF to polarize CD14+ monocytes to functional MDSCs, and rescued MDSC-mediated T-cell suppression in proliferation assay in vitro. On the other hand, conditioned medium from KRAS wild type cervical cancer cell line (HeLa) not expressing GM-CSF does not induce functional MDSCs.

Mix lymphocyte reaction was conducted as described above with pancreatic cancer (CAPAN-1) conditioned medium. FIG. 6B shows the results of the T-cell proliferation assay in terms of % of cells proliferating (left panel) and % of max (MFI) (signal detection of CFSE dilution in CD4+or CD8+ cells) by flow cytometry (right panel). As shown in FIG. 6B, CM-treated monocytes suppressed T-cell proliferation as compared to the control and addition of recombinant GM-CSF further suppressed T cell proliferation. Treatment with an anti-GM-CSFRα antibody (Ab) reduced the MDSC-mediated T cell suppression.

Next, mix lymphocyte reaction (n=2) was conducted as described above using colorectal cancer (SW-480) conditioned medium. In this particular experiment, anti-GM-CSFRα antibody was added to the polarized MDSCs on day 0, and anti-PD-L1 antibody (20 μg/mL) was added to the MLR on day 3. FIG. 6C shows that PD-L1 blockade on in vitro generated functional MDSCs blocks their ability to suppress T-cell proliferation. Notably, anti-GM-CSFRα was at least as effective as the anti-PD-L1 antibody in blocking PD-L1 on MDSCs and reversing the suppressive function of functional MDSCs. These data also suggest that PD-L1 expression associated with GM-CSF-dependent polarization of CD14+ monocytes to MDSCs is functionally significant in suppressing antitumor immunological responses.

### Example 6. KRAS-Mutant Cancer Cell Conditioned Medium Polarize Monocytes to Phenotypic MDSCs

This study illustrates that human monocytes cultured with conditioned medium from KRAS-mutant cancer cells expressing GM-CSF are polarized to phenotypic MDSCs.

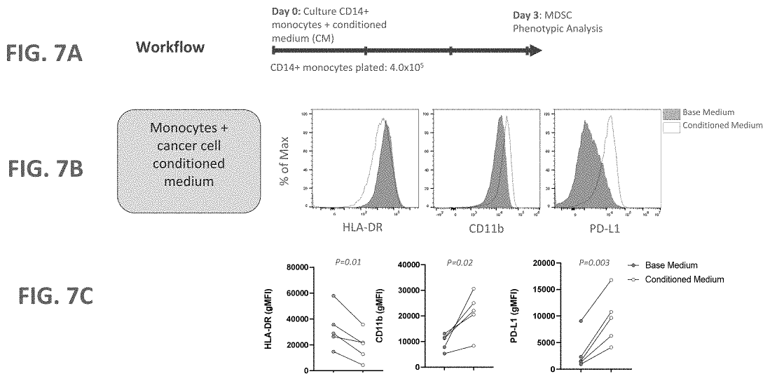

To generate KRAS-mutant tumor-conditioned media (CM), cell lines were plated and cultured according to methods known in the art. CD14+ monocytes were incubated with either base medium or conditioned medium from KRAS-mutant cancer cells for 3 days, followed by phenotypic analysis (HLA-DR, CD11b, and PD-L1) by flow cytometry, as shown in FIG. 7A. Low levels of HLA-DR biomarker is indicative of MDSC phenotype. Representative histogram overlays in FIG. 7B show decreased expression of HLA-DR and increased expression of CD11b and PD-L1. Similarly, relative expression, plotted as the geometric mean fluorescence intensity (gMFI), show a decrease in HLA-DR expression and an increase in CD11b, and PD-L1 expression when CD14+ monocytes were incubated with conditioned medium from KRAS-mutant cancer cells, as compared to CD+14 cells that were cultured in base medium (FIG. 7C). Results show that human monocytes cultured with conditioned medium from KRAS-mutant cancer cells expressing GM-CSF are polarized to phenotypic MDSCs, as illustrated by decreased expression of HLA-DR and increased expression of CD11b and PD-L1.

### Example 7. An Anti-GM-CSFRα Antibody Inhibits PD-L1 Expression Associated With Polarization of Monocytes to Phenotypic MDSCs in Vitro

The study in this example shows that a GM-CSF antagonist is sufficient to represses the expression of PD-L1 on monocytes treated with conditioned medium (CM) from KRAS-mutant cancer cell lines. Notably, a low dose of the GM-CSF antagonist (e.g., 0.1 μg/mL) was sufficient to achieve this effect.

As shown in Example 6, adding conditioned medium from KRAS-mutant cancer cell lines to the CD14+ monocytes increased the level of PD-L1 expression as compared to the baseline (base medium). In this study, the effect of an anti-GM-CSFRα antibody on PD-L1 expression was examined at various concentrations.

Conditioned medium from KRAS-mutant cancer cell lines containing GM-CSF (SW-480 and CAPAN-1) or conditioned medium from cancer cell line without GM-CSF (HeLa) were added to CD14+ monocytes with various concentrations (0.1 μg/mL, 1.0 μg/mL, 10.0 μg/mL, or 100 μg/mL) of anti-GM-CSFRα antibody. Base medium and conditioned medium without anti-GM-CSFRα antibody were used as controls. After three days of incubation, the MDSC cells were analyzed for expression of PD-L1.

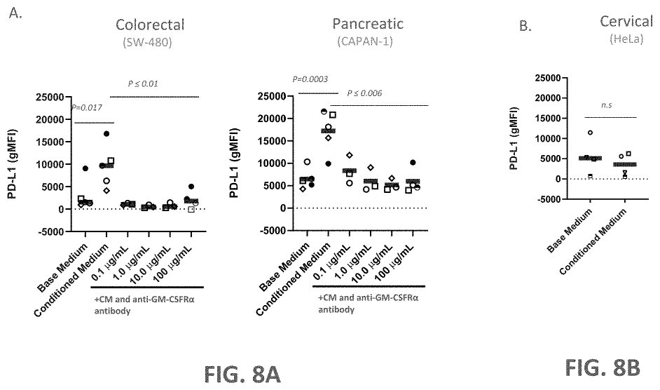

FIG. 8A shows that anti-GM-CSFRα antibody blocked PD-L1 upregulation on human monocytes cultured with conditioned medium from colorectal (SW-480) and pancreatic (CAPAN-1) KRAS-mutant cancer cells expressing GM-CSF and inhibited their polarization to phenotypic MDSCs. Notably, anti-GM-CSFRα antibody blocked PD-L1 upregulation even at a concentration of 0.1 μg/mL. Low GM-CSF-expressing human cervical carcinoma cell line (HeLa) conditioned medium does not induce phenotypic MDSCs with PD-L1 expression (FIG. 8B).

### Example 8. GM-CSF Antagonist Inhibits Tumor Growth in Vivo

The study in this example shows that a GM-CSF antagonist is able to inhibit tumor growth and provide survival advantage in vivo.

To establish a mouse model of pancreatic cancer that mimics the pathological features of pancreatic carcinoma, Pan02 cells were implanted subcutaneously in female C57BL/6 mice on day 0. On day 6, when tumors reached an average volume of 100 mm³, mice were randomly assigned to six different treatment group as shown in Table 2.

TABLE 2
Study design for efficacy of GM-CSF antibody in vivo

| Group | # mice | Treatment | Route | Schedule | Dosage |
|---|---|---|---|---|---|
| 1 | 10 | Vehicle + isotype control | IP + IP | (QDx5,2off)(x2) + | 0.2 mL/20 g + |
| | | | | (Q3Dx2,3off)(x2) | 10 mg/kg |
| 2 | 10 | Vehicle + anti-mPD-1 | IP + IP | (QDx5,2off)(x2) + | 0.2 mL/20 g + |
| | | | | (Q3Dx2,3off)(x2) | 10 mg/kg |
| 3 | 10 | anti-GM-CSFRα | IP + IP | Q2Dx7 + | 20 mg/kg + |
| | | antibody + isotype | | (Q3Dx2,3off)(x2) | 10 mg/kg |
| | | control | | | |
| 4 | 10 | anti-GM-CSFRα | IP + IP | (QDx5,2off)(x2) + | 20 mg/kg + |
| | | antibody + isotype | | (Q3Dx2,3off)(x2) | 10 mg/kg |
| | | control | | | |
| 5 | 10 | anti-GM-CSFRα | IP + IP | Q2Dx7 + | 20 mg/kg + |
| | | antibody + anti-mPD-1 | | (Q3Dx2,3off)(x2) | 10 mg/kg |
| 6 | 10 | anti-GM-CSFRα | IP + IP | (QDx5,2off)(x2) + | 20 mg/kg + |
| | | antibody + anti-mPD-1 | | (Q3Dx2,3off)(x2) | 10 mg/kg |

Mice in group 1 were treated with daily intraperitoneal (IP) injection with vehicle (negative control) for 5 days per week, with a 2-day treatment holiday, for two weeks, and IP injection with isotype control antibody twice per week, dosing every third day, with a 3-day treatment holiday, for two weeks. Mice in group 2 were treated with daily IP injection with vehicle for 5 days per week, with a 2-day treatment holiday, for two weeks, and IP injection with anti-mPD-1 antibody twice per week, dosing every third day, with a 3-day treatment holiday, for two weeks. Mice in group 3 were treated with IP injection with anti-GM-CSFRα antibody every other day for two weeks, and IP injection with isotype control antibody twice per week, dosing every third day, with a 3-day treatment holiday, for two weeks. Mice in group 4 were treated with daily IP injection with anti-GM-CSFRα antibody for 5-days per week, with a 2-day treatment holiday, for two weeks, and IP injection with isotype control antibody twice per week, dosing every third day, with a 3-day treatment holiday, for two weeks. Mice in group 5 were treated with IP injection with anti-GM-CSFRα antibody every other day for two weeks, and IP injection with anti-mPD-1 antibody twice per week, dosing every third day, with a 3-day treatment holiday, for two weeks. Mice in group 6 were treated with daily IP injection with anti-GM-CSFRα antibody for 5 days per week, with a 2-day treatment holiday, for two weeks, and IP injection with anti-mPD1 antibody twice per week, dosing every third day, with a 3-day treatment holiday, for two weeks. 20 mg/kg dose for anti-GM-CSFRα antibody, 10 mg/kg dose for isotype control antibody, 10 mg/kg for anti-mPD-1 antibody, and 0.2 mL/20g dose for vehicle were used for all treatment groups, as shown in Table 2.

Tumor measurements were taken every other day throughout the study. Primary end-point for the study was delay in tumor growth and overall survival rate.

### Inhibition of Tumor Growth

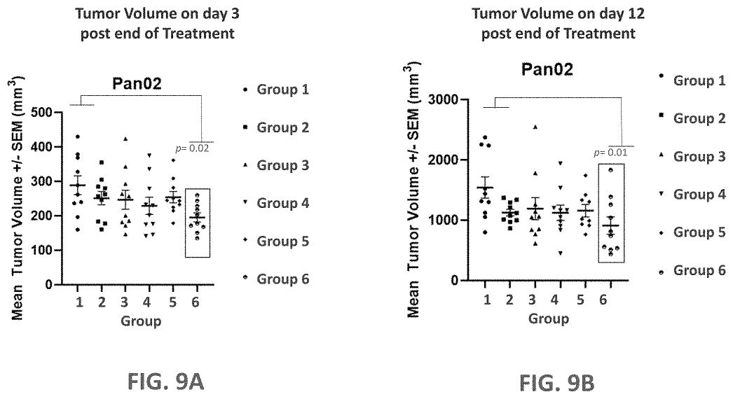

To examine if anti-GM-CSFRα antibody inhibits tumor growth in mice with pancreatic carcinoma, tumor volume of each mouse was measured 3 and 12 days after the end of the treatment and plotted as shown in FIG. 9A and FIG. 9B, respectively. As shown in FIG. 9A, there is a directional improvement in inhibition of tumor growth when mice were treated with anti-GM-CSFRα antibody alone or in combination (Groups 3, 4 and 5 vs. Group 1). Additionally, combination therapy with anti-GM-CSFRα antibody, dosed daily for 5 days, for 2 weeks, with anti-mPD-1 antibody (Group 6), results in statistically significant tumor growth inhibition compared to a control (Group 1), and single agents (Groups 2, 3, and 4). FIG. 9B also shows the same trend when tumor volumes were measured 12 days post the end of the treatment.

### Survival Advantage

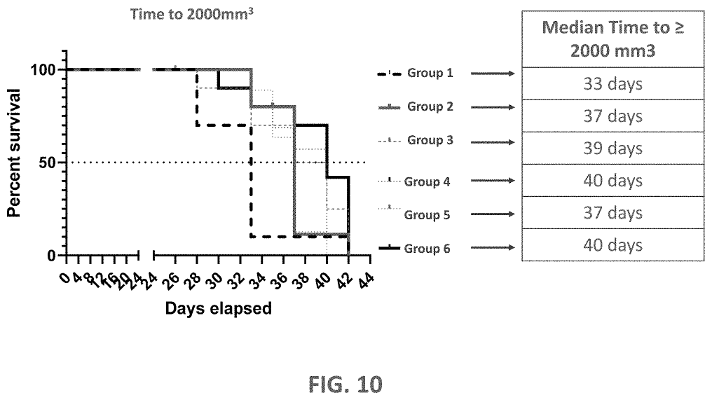

To examine if anti-GM-CSFRα antibody provides survival advantage to mice with pancreatic carcinoma, tumor volume of each mouse was measured on indicated days as shown in FIG. 10. When tumor volume exceeded 2000 mm³, mice were sacrificed. The time to reach tumor volume of 2000 mm³ or greater was assessed and portrayed in the Kaplan-Meier plot as shown in FIG. 10. The time to tumor volume equal to or greater than 2000 mm³ was longer in Group 3 and Group 4, which were treated with anti-GM-CSFRα antibody alone, as compared to a control group (Group 1) or Group 2, which was treated with anti-mPD-1 antibody only. Additionally, the time to tumor volume equal to or greater than 2000 mm³ was longer in Group 6, which was treated with anti-GM-CSFRα antibody, dosed daily for 5 days for 2 weeks, and anti-mPD-1 antibody, as compared to a control group (Group 1) or Group 2, which was treated with anti-mPD-1 antibody only. Overall, the data show that treatment with anti-GM-CSFRα antibody alone or in combination with anti-PD1 results in survival advantage compared to control or treatment with anti-PD1 antibody alone.

### Equivalents

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A method of treating cancer with a KRAS mutation comprising administering a GM-CSF antagonist to the patient in need of treatment, wherein the administration of the GM-CSF antagonist results in inhibition of an immunosuppressive activity of myeloid-derived suppressor cells (MDSCs).

2. A method of inhibiting immunosuppressive activity of myeloid-derived suppressor cells (MDSCs) in a patient suffering from cancer with a KRAS mutation comprising administering a GM-CSF antagonist to the patient.

3. A method of enhancing immune response for cancer treatment comprising administering a GM-CSF antagonist to a patient receiving a cancer treatment, wherein the patient is suffering from cancer with one or more mutations in KRAS, NRAS, or JAK2, wherein the immune response is increased as compared to a control.

4. The method of claim 3, wherein the cancer with one or more mutations in KRAS, NRAS, PTPN11 or JAK2 is chronic myelomonocytic leukemia (CMML).

5. The method of claim 3, wherein the cancer with one or more mutations in KRAS, NRAS, PTPN11 or JAK2 is juvenile myelomonocytic leukemia (JMML).

6. (canceled)

7. The method of claim 3, wherein the control is indicative of the immune response level in the patient prior to the administration of GM-CSF antagonist.

8-10. (canceled)

11. A method of suppressing PD-L1 in a patient suffering from cancer with one or more mutations in KRAS, NRAS, PTPN11 and/or JAK2, comprising administering a GM-CSF antagonist to a patient in need of treatment as compared to a control.

12. The method of claim 11, wherein the administering the GM-CSF antagonist decreases a level of PD-L1 in the patient.

13-14. (canceled)

15. The method of claim 12, wherein the level of PD-L1 in the patient is decreased by at least 10%, 20%, 30%, 50%, 60%, 70%, 80% or 90% as compared to the control.

16-29. (canceled)

30. The method of claim 1, wherein the method further comprises administering at least one other cancer therapy to the patient, and wherein the patient has received a treatment with the GM-CSF antagonist prior to the administration of the other cancer therapy.

31. The method of claim 30, wherein the other cancer therapy is an ICI.

32-33. (canceled)

34. The method of claim 31, wherein the ICI is an anti-PD-L1 antibody.

35-41. (canceled)

42. The method of claim 1, wherein the GM-CSF antagonist is an anti-GM-CSF receptor antibody or a fragment thereof.

43. The method of claim 42, wherein the anti-GM-CSF receptor antibody or a fragment thereof is an anti-GM-CSFRα antibody or a fragment thereof.

44-45. (canceled)

46. The method of claim 42, wherein the anti-GM-CSFRα antibody is mavrilimumab.

47. The method of claim 43, wherein the anti-GM- CSFRα antibody a fragment thereof comprises a light chain complementary-determining region 1 (LCDR1) defined by SEQ ID NO: 6, a light chain complementary-determining region 2 (LCDR2) defined by SEQ ID NO: 7, and a light chain complementary-determining region 3 (LCDR3) defined by SEQ ID NO: 8; and a heavy chain complementary-determining region 1 (HCDR1) defined by SEQ ID NO: 3, a heavy chain complementary-determining region 2 (HCDR2) defined by SEQ ID NO: 4, and a heavy chain complementary-determining region 3 (HCDR3) defined by SEQ ID NO: 5.

48. The method of claim 31, wherein the administration of the GM-CSF antagonist and/or the ICI results in reduced level of MDSCs in the patient as compared to a control.

49-58. (canceled)

59. The method claim 1, wherein the GM-CSF antagonist is administered between 2 mg/kg-10 mg/kg.

60-64. (canceled)

65. A pharmaceutical composition for treating cancer with a KRAS mutation comprising a GM-CSF antagonist and an ICI.

66-75. (canceled)

76. A kit for treating cancer with one or more mutation in KRAS, NRAS, and/or JAK2 comprising a pharmaceutical composition of claim 65.

77-88. (canceled)
